(19) United States
(12) Patent Application Publication
(10) Pub. No.: US 2020/0123608 A1
(43) Pub. Date: Apr. 23, 2020

(54) RNA SEQUENCES FOR BODY FLUID IDENTIFICATION

(71) Applicant: INSTITUTE OF ENVIRONMENTAL SCIENCE AND RESEARCH LIMITED, Porirua (NZ)

(72) Inventors: Meng-Han LIN, Auckland (AU); Patricia Pearl ALBANI, Auckland (NZ); Rachel Ingrid FLEMING, Auckland (NZ)

(21) Appl. No.: 16/339,867

(22) PCT Filed: Oct. 5, 2017
(86) PCT No.: PCT/NZ2017/050128

§ 371 Date: Apr. 5, 2019

(30) Foreign Application Priority Data
Oct. 5, 2016 (NZ) 724876

(51) Int. Cl. C12Q 1/6881

(52) U.S. Cl. CPC C12Q 1/6881; C12Q 2600/16; C12Q 2600/158

(57) ABSTRACT

The present invention provides for the identification of novel RNA sequences and methods of utilizing such sequences in identifying the tissue origin of biological samples. Specifically, the invention provides for RNA sequences associated with a method of testing to establish whether a biological sample is of circulatory blood, spermatozoa, seminal fluid or menstrual fluid origin.

### TECHNICAL FIELD The technical field is the detection of RNA sequences, and the use of these sequences for identification and typing of samples, in particular samples containing degraded RNA.

### BACKGROUND

The ability to accurately detect and quantify RNA abundance is a fundamental capability in molecular biology. The broad set of RNA detection methods currently available range from non-amplification methods (in situ hybridisation, microarray and NanoString nCounter), to amplification (PCR) based methods (reverse transcriptase PCR (RT-PCR) and quantitative reverse transcriptase PCR (qRT-PCR)). With the exception of RNAseq (next generation sequencing, also referred to as second generation sequencing or massively parallel sequencing), a key prerequisite of all RNA detection technology is prior knowledge of the target RNA sequence. This targeting is facilitated by oligonucleotide sequences in both non-amplification methods (probe) and amplification-based methods (primers).

Methods for PCR primer design are always evolving [1, 2] but remain based around the core criteria of specificity, thermodynamics, secondary structure, dimerisation and amplicon length [3-7]. In addition to these criteria, RT-PCR primer design (for RNA amplification) also considers exon boundary coverage to ensure amplification of only cDNA and avoid amplification of genomic DNA [8]. Amongst other experimental factors [9-14], it is widely acknowledged that PCR primer design has critical implications to target amplification, detection and quantification [3, 8, 11, 15-18].

Whilst improvements to primer design can yield performance improvements, the target molecule must also be considered. RNA is unstable and easily degraded [19-22]. Conventional methodology recommends sample RNA integrity (RIN) to be at least RIN 8 or above to ensure proper performance [23-26]. RIN values range from 10 (intact) to 1 (totally degraded). The gradual degradation of RNA is reflected by a continuous shift towards shorter RNA fragments the more degraded the RNA is. In this context shorter means that the RNA fragments are not as long as non-degraded RNA and over time the RNA fragments break down into smaller and smaller fragments.

A degree of degradation is unavoidable in situations where real-world samples must be analysed—forensic, clinical, FFPE and environmental sampling. The detrimental effects of RNA degradation on RNA detection and quantification are well documented [24, 27-30]. Currently there is no clear solution to this problem except to avoid analysing degraded RNA.

Here the inventors have established the identification of blood, semen (with or without spermatazoa), and menstrual fluid by detection of specific RNA sequences.

It is an object of the invention is to provide improved methods and/or materials for specific detection of RNA sequences in samples that have been subject to degradation. It is a further or alternate object of the invention to provide a method and/or materials for specific detection of RNA sequences in samples and/or at least to provide the public with a useful choice.

### SUMMARY OF THE INVENTION

The present invention provides methods for design, production and use of probes and primers that are directed to stable regions of RNA of interest. The methods involve the use of next generation sequencing to identify stable regions of RNA. Probes or primers are then designed that will hybridise to the identified stable regions.

RNA detection assays (including amplification—or non-amplification—based methods) are then designed that include sequences corresponding to the stable regions for identification and typing of samples containing RNA.

When RNA next generation sequencing data shows a higher number of sequencing reads aligned to a particular region of a given RNA, then this region is more stable, or less degraded, than regions of the RNA with fewer, or no, aligned sequencing reads. RNA regions of lower sequencing read coverage were postulated to indicate regions where the transcript has degraded. Targeting the stable regions for primer design, allows improved detection of the RNA relative to that shown when standard primer design approaches are used.

The inventors have shown that this invention is particularly useful for detection of RNA sequence of interest in forensic samples. Detection of such RNA sequences, or RNA marker sequences, is useful in identification or typing or any given forensic sample. The invention is particularly useful for detection of such RNA marker sequences in samples that have been subjected to degradation, as is often the case for forensic samples.

### METHODS

In one aspect the invention provides a method for the detection an RNA sequence in a sample, the method including the steps:
a) providing a sample, and
b) detecting the RNA sequence using at least one primer selected from the group comprising a sequence complementary to a part of SEQ ID NO:1 to SEQ ID NO:95 or compliment of anyone thereof, or a sequence comprising SEQ ID NO:96 to SEQ ID NO: 107, or a complement of any one thereof; or at least one probe selected from the group comprising a sequence that is complementary to a part of SED ID NO:1 to SEQ ID NO:95 or compliment of anyone thereof.

Preferably the RNA sequence has been identified using RNA sequencing of the sample.

Preferably the RNA sequence has been identified as a region in the RNA sequence which has more aligned sequencing reads than another region, or regions, of the same RNA sequence.

Preferably the RNA sequence is selected from the group comprising SEQ ID NO:1 to SEQ ID NO:95 or a compliment of anyone thereof.

Preferably the sample is a biological tissue sample.

Preferably the sample is a solid sample.

Preferably the sample is a liquid sample.

Preferably the sample is a forensic sample.

Preferably the forensic sample is selected from the group comprising blood, semen (with or without spermatazoa), and menstrual fluid.

Preferably the RNA is extracted from the sample prior to the detecting step.

Preferably the RNA sequence is detected directly.

Preferably the RNA sequence is detected indirectly.

Preferably the RNA sequence is detected indirectly by detection of a complementary DNA (cDNA) corresponding to the RNA sequence.

In one aspect the invention provides a method of typing a sample including RNA, the method including the steps:
a) providing a sample, and
b) detecting one or more stable RNA sequences in the sample using at least one primer selected from the group comprising a sequence complementary to a part of SEQ ID NO:1 to SEQ ID NO:95 or compliment of anyone thereof, or a sequence comprising SEQ ID NO:96 to SEQ ID NO: 107, or a complement of any one thereof; or at least one probe selected from the group comprising a sequence that is complementary to a part of SED ID NO:1 to SEQ ID NO:95 or compliment of anyone thereof;

wherein the stable RNA sequence is specific for the type of sample.

Preferably the stable region of the RNA sequence has been identified using RNA sequencing of the sample.

Preferably the stable region of the RNA sequence has been identified as a region in the RNA sequence which has more aligned sequencing reads than another region, or regions, of the same RNA sequence.

Preferably the stable region is selected from the group comprising SEQ ID NO:1 to SEQ ID NO:95 or a compliment of anyone thereof.

Preferably the sample is a biological tissue sample.

Preferably the sample is a solid sample.

Preferably the sample is a liquid sample.

Preferably the sample is a forensic sample.

Preferably the forensic sample is selected from the group comprising blood, semen (with or without spermatazoa), and menstrual fluid.

Preferably the RNA is extracted from the sample prior to the detecting step.

Preferably the RNA sequence is detected directly.

Preferably the RNA sequence is detected indirectly.

Preferably the RNA sequence is detected indirectly by detection of a complementary DNA (cDNA) corresponding to the RNA sequence.

In one embodiment the invention provides a method of typing a sample including degraded RNA, the method including the steps:
a) providing a sample, and
b) detecting one or more stable RNA sequences in the sample using at least one primer selected from the group comprising a sequence complementary to a part of SEQ ID NO:1 to SEQ ID NO:95 or compliment of anyone thereof, or a sequence comprising SEQ ID NO:96 to SEQ ID NO: 107, or a complement of any one thereof; or at least one probe selected from the group comprising a sequence that is complementary to a part of SED ID NO:1 to SEQ ID NO:95 or compliment of anyone thereof;

wherein the stable RNA sequence is specific for the type of sample; and,

wherein detecting the stable RNA region indicates the type of sample.

Preferably the stable region of the RNA sequence has been identified using RNA sequencing of the sample.

Preferably the stable region of the RNA sequence has been identified as a region in the RNA sequence which has more aligned sequencing reads than another region, or regions, of the same RNA sequence.

Preferably the stable region is selected from the group comprising SEQ ID NO:1 to SEQ ID NO:95 or a compliment of anyone thereof.

Preferably the sample is a biological tissue sample.

Preferably the sample is a solid sample.

Preferably the sample is a liquid sample.

Preferably the sample is a forensic sample.

Preferably the forensic sample is selected from the group comprising blood, semen (with or without spermatazoa), and menstrual fluid.

Preferably the RNA is extracted from the sample prior to the detecting step.

Preferably the RNA sequence is detected directly.

Preferably the RNA sequence is detected indirectly.

Preferably the RNA sequence is detected indirectly by detection of a complementary DNA (cDNA) corresponding to the RNA sequence.

### Detection with Primer

In one embodiment the RNA sequence is detected using a primer.

Preferably the RNA sequence is detected using two primers.

Preferably both of the primers correspond to, are complementary to, or are capable of hybridising to, a sequence within the stable region.

Preferably both of the primers correspond to, are complementary to, or are capable of hybridising to, a sequence within a sequence selected from SEQ ID NO:1 to SEQ ID NO:95 or compliment of anyone thereof.

Preferably the primer is selected from the group comprising SEQ ID NO:96 to SEQ ID NO: 107, or a complement of anyone thereof.

In these embodiments the primers are used to amplify the part of the stable region bound by the primers.

In one embodiment amplification is by a polymerase chain reaction (PCR) method.

In one embodiment the PCR method is selected from standard PCR, reverse transcriptase (RT)-PCR, and quantitative reverse transcriptase PCR (qRT-PCR)

### Detection with Probe

In a further embodiment the RNA sequence is detected using a probe.

Preferably the probe corresponds to, or is complementary to, a sequence within the stable region.

Preferably the probe corresponds to, is complementary to, or is capable of hybridising to, a sequence within a sequence selected from SEQ ID NO:1 to SEQ ID NO:95 or compliment of anyone thereof.

### Sample

In one embodiment the sample is a biological tissue sample.

In a further embodiment the sample is a solid sample. In a further embodiment the sample is a liquid sample.

In a preferred embodiment the sample is a forensic sample.

Preferably the forensic sample is selected from the group comprising blood, semen (with or without spermatazoa), and menstrual fluid.

### Markers Within Sample

In one embodiment the RNA sequence is encoded by a marker gene specific for the type of sample.

That is, the expression of the RNA sequence, or presence of the RNA sequence, in the sample, is diagnostic for the type of sample.

In one embodiment, when the sample is circulatory blood, the marker gene is selected from:
Hemoglobin delta (HBD),
Solute carrier family 4 (anion exchanger), member 1 (Diego blood group) (SLC4A1).

In a further embodiment when the sample is spermatazoa, the marker gene is Transition protein 1 (during histone to protamine replacement) (TNP1).

In a further embodiment when the sample is seminal fluid, the marker gene is Kallikrein-related peptidase 2 (KLK2).

In a further embodiment when the sample is menstrual fluid, the marker gene is selected from:
Matrix metallopeptidase 3 (MMP3), and
Stanniocalcin 1 (STC1).

In a further embodiment the stable region of the RNA sequence corresponds to the cDNA sequence of any one of SEQ ID NO:1 to 92.

In a further aspect the invention provides a nucleotide sequence comprising at least 5 nucleotides with at least 70% identity to a sequence selected from SEQ ID NO:1 to SEQ ID NO:95.

In a further aspect the invention provides a nucleotide sequence comprising at least 5 nucleotides of a sequence selected from SEQ ID NO:1 to SEQ ID NO:95 or a compliment thereof.

In a further aspect the invention provides a nucleotide sequence comprising at least 10 nucleotides with at least 70% identity to a sequence selected from SEQ ID NO:1 to SEQ ID NO:95 or a compliment thereof.

In a further aspect the invention provides a nucleotide sequence comprising at least 10 nucleotides of a sequence selected from SEQ ID NO:1 to SEQ ID NO:95 or a compliment thereof.

In a further aspect the invention provides a nucleotide sequence selected from any one of SEQ ID NO:1 to SEQ ID NO:95

In a further aspect the invention provides a nucleotide sequence selected from anyone of SEQ ID NO:96 to SEQ ID NO: 107, or a complement of any one thereof.

In a further aspect the invention provides the use of a nucleotide sequence defined above in the typing of a sample including RNA.

### Primers

In a further embodiment detection involves use of a primer capable of hybridising to the stable region of the RNA sequence, or a cDNA corresponding to the stable region or a complement thereof.

In a further embodiment detection involves use of a primer comprising a sequence of at least 5 nucleotides with at least 70% identity to any part of the sequence of any one of SEQ ID NO:1 to 95 or a complement thereof.

In a further embodiment the primer consists of a sequence of at least 5 nucleotides with at least 70% identity to the sequence of any one of SEQ ID NO:1 to 95 or a complement thereof.

In a further embodiment the primer comprises a sequence of at least 5 nucleotides of the sequence of any one of SEQ ID NO:1 to 95, or a complement thereof.

In a further embodiment the primer consists of a sequence of at least 5 nucleotides of the sequence of any one of SEQ ID NO:1 to 95, or a complement thereof.

In a further embodiment the primer comprises a selected from the group comprising SEQ ID NO:96 to SEQ ID NO: 107, or a complement of any one thereof.

In a further embodiment the primer consists of a sequence selected from the group comprising SEQ ID NO:96 to SEQ ID NO: 107, or a complement of any one thereof.

In a further embodiment the primer is selected from the group comprising SEQ ID NO:96 to SEQ ID NO: 107, or a complement of any one thereof.

In a further embodiment the primer includes an attached label or tag.

### Probes

In a further embodiment detection involves use of a probe capable of hybridising to the stable region of the RNA sequence, or a cDNA corresponding to the stable region or a complement thereof.

In a further embodiment detection involves use of a probe comprising a sequence of at least 10 nucleotides with at least 70% identity to any part of the sequence of any one of SEQ ID NO:1 to 95 or a complement thereof.

In a further embodiment the probe consists of a sequence of at least 5 nucleotides with at least 70% identity to the sequence of any one of SEQ ID NO:1 to 95, or a complement thereof.

In a further embodiment the probe comprises a sequence of at least 5 nucleotides of the sequence of any one of SEQ ID NO:1 to 95, or a complement thereof.

In a further embodiment the probe consists of a sequence of at least 5 nucleotides of the sequence of any one of SEQ ID NO:1 to 95, or a complement thereof.

In a further embodiment the probe includes an attached label or tag.

### Typing a Sample

In a further aspect the invention provides a method of typing a sample, the method comprising the steps of detecting an RNA sequence in a sample by a method of the invention, wherein detecting the RNA sequence marker indicates the type of sample.

The method may involve using just one pair of primers, or a single probe, to type the sample. Alternatively multiple pairs of primers, or multiple probes, may be used.

### Typing Sample by Multiplex PCR

In one embodiment multiplex PCR is performed with multiple primers, at least one of which is diagnostic for the type of sample.

Preferably multiplex PCR is performed using at least 4, more preferably at least 5, more preferably at least 6, more preferably at least 7, more preferably at least 8, more preferably at least 9, more preferably at least 10, more preferably at least 11, more preferably at least 12, more preferably at least 13, more preferably at least 14, more preferably at least 15, more preferably at least 16, more preferably at least 17, more preferably at least 18, more preferably at least 19, more preferably at least 20, more preferably at least 21, more preferably at least 22, more preferably at least 23, more preferably at least 24, more preferably at least 25, more preferably at least 26, more preferably at least 27, more preferably at least 28, more preferably at least 29, more preferably at least 30 primers of the invention.

In a preferred embodiment, the method of the invention results in amplification of a product, or a hybridisation event, that would not occur in nature, or in the absence of the method of the invention.

### PRODUCTS

### Primers

In a further embodiment the invention provides a primer capable of hybridising to the stable region of the RNA sequence, or a cDNA corresponding to the stable region or a complement thereof.

In a further embodiment the invention provides a primer comprising a sequence of at least 5 nucleotides with at least 70% identity to any part of the sequence of any one of SEQ ID NO:1 to 95 or a complement thereof.

In a further embodiment the primer consists of a sequence of at least 5 nucleotides with at least 70% identity to the sequence of any one of SEQ ID NO:1 to 95, or a complement thereof.

In a further embodiment the primer comprises a sequence of at least 5 nucleotides of the sequence of any one of SEQ ID NO:1 to 95, or a complement thereof.

In a further embodiment the primer consists of a sequence of at least 5 nucleotides of the sequence of any one of SEQ ID NO:1 to 95, or a complement thereof.

In a further embodiment the primer comprises a selected from the group comprising SEQ ID NO:96 to SEQ ID NO: 107, or a complement of any one thereof.

In a further embodiment the primer consists of a sequence selected from the group comprising SEQ ID NO:96 to SEQ ID NO: 107, or a complement of any one thereof.

In a further embodiment the primer is selected from the group comprising SEQ ID NO:95 to SEQ ID NO: 107, or a complement of any one thereof.

In a further embodiment the primer includes an attached label or tag.

In a further embodiment the labelled or tagged primer is not found in nature.

The primers of the invention can be used on microarrays or chips or like products for the detection of RNA sequences.

### Kit of Primers

In a further embodiment the invention provides a kit comprising at least one primer of the invention.

Preferably the kit comprises at least 2, more preferably at least 3, more preferably at least 4, more preferably at least 5, more preferably at least 6, more preferably at least 7, more preferably at least 8, more preferably at least 9, more preferably at least 10, more preferably at least 11, more preferably at least 12, more preferably at least 13, more preferably at least 14, more preferably at least 15, more preferably at least 16, more preferably at least 17, more preferably at least 18, more preferably at least 19, more preferably at least 20, more preferably at least 21, more preferably at least 22, more preferably at least 23, more preferably at least 24, more preferably at least 25, more preferably at least 26, more preferably at least 27, more preferably at least 28, more preferably at least 29, more preferably at least 30 primers of the invention. In one embodiment the kit also comprises instructions for use.

### Probes

In a further embodiment the invention provides a probe capable of hybridising to the stable region of the RNA sequence, or a cDNA corresponding to the stable region or a complement thereof.

In a further embodiment the invention provides a probe comprising a sequence of at least 10 nucleotides with at least 70% identity to any part of the sequence of any one of SEQ ID NO:1 to 95 or a complement thereof.

In a further embodiment the probe consists of a sequence of at least 10 nucleotides with at least 70% identity to the sequence of any one of SEQ ID NO:1 to 95, or a complement thereof.

In a further embodiment the probe comprises a sequence of at least 10 nucleotides of the sequence of any one of SEQ ID NO:1 to 95, or a complement thereof.

In a further embodiment the probe consists of a sequence of at least 10 nucleotides of the sequence of any one of SEQ ID NO:1 to 95, or a complement thereof.

In a further embodiment the probe includes an attached label or tag.

In a further embodiment the labelled or tagged probe is not found in nature.

The primers of the invention can be used on microarrays or chips or like products for the detection of RNA sequences.

### Kit of Probes

In a further embodiment the invention provides a kit comprising at least one probe of the invention.

Preferably the kit comprises at least 2, more preferably at least 3, more preferably at least 4, more preferably at least 5, more preferably at least 6, more preferably at least 7, more preferably at least 8, more preferably at least 9, more preferably at least 10, more preferably at least 11, more preferably at least 12, more preferably at least 13, more preferably at least 14, more preferably at least 15, more preferably at least 16, more preferably at least 17, more preferably at least 18, more preferably at least 19, more preferably at least 20, more preferably at least 21, more preferably at least 22, more preferably at least 23, more preferably at least 24, more preferably at least 25, more preferably at least 26, more preferably at least 27, more preferably at least 28, more preferably at least 29, more preferably at least 30 probes of the invention.

In one embodiment the kit also comprises instructions for use.

### MicroArrays

In another aspect the invention provides a microarray comprising a sequence of at least 5 nucleotides with at least 70% identity to any part of the sequence of any one of SEQ ID NO:1 to SEQ ID NO:95 or a complement thereof.

In another aspect the invention provides a microarray comprising a sequence of at least 5 nucleotides of a sequence of any one of SEQ ID NO:1 to SEQ ID NO:95 or a complement thereof.

In another aspect the invention provides a microarray comprising a sequence of at least 10 nucleotides of a sequence with at least 70% identify to any part of the sequence of any one of SEQ ID NO:1 to SEQ ID NO:95 or a complement thereof.

In another aspect the invention provides a microarray comprising a sequence of at least 10 nucleotides of a sequence of any one of SEQ ID NO:1 to SEQ ID NO:95 or a complement thereof.

Preferably the sequence comprises at least 5, more preferably at least 10, more preferably at least 15, more preferably at least 20, more preferably at least 25, more preferably at least 30, more preferably at least 35, more preferably at least 40, more preferably at least 45, more preferably at least 50, more preferably at least 55, more preferably at least 60, more preferably at least 65, more preferably at least 70, more preferably at least 75, more preferably at least 80, more preferably at least 85, more preferably at least 90, more preferably at least 95, more preferably at least 100, more preferably at least 120, more preferably at least 140, more preferably at least 160, more preferably at least 180, more preferably at least 200, more preferably at least 240, more preferably at least 250 nucleotides of the sequences of the invention.

Those skilled in the art would understand how to select the appropriate probes or primers for detecting any of the listed markers, based on the information in the Sequene Listing, and elsewhere in the specification.

It will be understood to those skilled in the art that a probe or primer can be produced that can hybridise to any part of a stable region. The probes and primers mentioned herein are given as examples only to demonstrate that the stable regions can be used to identify and type degraded RNA. Any primer or probe that is complementary to the stable region would be suitable in the methods of the invention.

Those skilled in the art will understand the relationship between marker genes, the mRNA encoded by the marker genes, and the stable regions within the mRNA. Those skilled in the art will understand that the sequences presented are DNA sequences corresponding to the mRNA or stable regions within the mRNA.

### DETAILED DESCRIPTION OF THE INVENTION

In this specification where reference has been made to patent specifications, other external documents, or other sources of information, this is generally for the purpose of providing a context for discussing the features of the invention. Unless specifically stated otherwise, reference to such external documents is not to be construed as an admission that such documents, or such sources of information, in any jurisdiction, are prior art, or form part of the common general knowledge in the art.

The term “comprising” as used in this specification and claims means “consisting at least in part of”; that is to say when interpreting statements in this specification and claims which include “comprising”, the features prefaced by this term in each statement all need to be present but other features can also be present. Related terms such as “comprise” and “comprised” are to be interpreted in similar manner. However, in preferred embodiments comprising can be replaced with consisting.

As used here, the term “RNA” means messenger RNA, small RNA, microRNA, non-coding RNA, long non-coding RNA, small non-coding RNA, ribosomal RNA, small nucleolar RNA, transfer RNA and all other RNA species and sequences.

As used herein, the term “stable region” means a region or regions in an RNA sequence which has more aligned sequencing reads than another region, or regions, of the same RNA sequence.

As used herein the term “degraded RNA” refers to is RNA that is no longer intact. In other words, the theoretical full length RNA, as annotated or predicted in sequence databases, is no longer intact. The full length RNA may be fragmented and/or some nucleotides are no longer present. This may occur at any position along the RNA sequence.

One measure of the level of degradation in an RNA sequence is the RNA integrity (RIN) value. RIN values range from 10 (fully intact) to 1 (totally degraded). Conventional methodology recommends sample RNA integrity (RIN) to be at least RIN 8 or above to ensure proper performance of RNA analysis as previously discussed.

Another measure of degradation in an RNA sequence is DV200 (Zhao, Shanrong, Baohong Zhang, Ying Zhang, William Gordon, Sarah Du, Theresa Paradis, Michael Vincent, and David von Schack. “Bioinformatics for RNA-Seq Data Analysis.” BIOINFORMATICS-UPDATED FEATURES AND APPLICATIONS (2016): 125).

The inventors stress that how the level of RNA degradation is measured is not essential and the invention lies in the ability to detect degraded RNA.

The inventors have found specific stable regions in RNA specific to sample types. These stable regions can be targeted to type samples using primers and probes. The stable regions can be used to type samples having RIN values of less than 8 but also, as those stable regions will also be present in other equivalent samples having RIN values of greater than 8, the stable regions can be used to type samples if they have RIN values of greater than 8 as well.

The present invention provides improved materials and methods for detecting RNA sequences in samples. The method involves using RNA sequencing to identify stable regions of RNA of interest on the basis of RNA sequencing data showing multiple aligned reads over the regions.

The method of the invention then involves producing probes or primers targeting the stable regions. The method allows for improved detection of such RNA sequences, particularly in samples in which the RNA is, or has been, subjected to degradation.

### RNA Degradation

Whilst improvements to primer or probe design can yield performance improvements in amplification and hybridisation methods, the target molecule must also be considered. RNA is unstable and easily degraded [19-22]. Conventional methodology recommends sample RNA integrity (RIN) to be at least RIN 8 or above to ensure proper performance [23-26].

A degree of degradation is unavoidable in situations where real-world samples must be analysed—forensic, clinical, FFPE and environmental sampling. The detrimental effects of RNA degradation on RNA detection and quantification are well documented [24, 27-30].

The methods and materials of the invention allow for improved detection of RNA sequences of interest, particularly when RNA samples have been degraded. This allows typing of samples that contain that degraded RNA, including samples having a RIN value less than 8. This is particularly surprising as prior to the present invention it was generally considered that detection and typing of degraded RNA sequences where RIN was less than 8, was not able to be achieved to an acceptable performance value.

RIN values range from 10 (intact) to 1 (totally degraded). The gradual degradation of RNA is reflected by a continuous shift towards shorter RNA fragments the more degraded the RNA is. Where the RIN value is less than 1, this signifies that RNA is degraded beyond detection.

While the inventors have found that while the probes and primers of the invention are useful in detecting and typing the source of degraded RNA including RNA having a RIN value less than 8, the probes and primers of the invention can also be used to detect and type the source of RNA having a RIN value of 8-10. That is, the primers and probes of the invention also allow the detection and typing of RNA irrespective of the RIN value.

In one embodiment the methods of the invention works, or allow for RNA marker detection, when RNA integrity (RIN) is less than RIN 8, more preferably less than RIN 7, more preferably less than RIN 6, more preferably less than RIN 5, more preferably less than RIN 4, more preferably less than RIN 3, more preferably less than RIN 2, more preferably less that than 1. The inventors have also found that the methods of the invention can be used to type RNA where RIN is undetermined (beyond detection).

### Applications for the Methods and Materials of the Invention

The methods and materials of the invention may be applied to any process involving detection of RNA, particularly in situations where degradation of target RNA is a problem.

The broad set of RNA detection methods currently available range from non-amplification methods (in situ hybridisation, microarray and NanoString nCounter), to amplification (PCR) based methods (reverse transcriptase PCR (RT-PCR) and quantitative reverse transcriptase PCR (qRT-PCR), next generation sequencing (massively parallel sequencing/high throughput sequencing), and RNA-aptamers.

### In Situ Hybridisation

In situ hybridization (ISH) is a type of hybridization that uses a labelled complementary DNA or RNA strand (i.e., probe) to localize a specific DNA or RNA sequence in a portion or section of tissue (in situ), or, if the tissue is small enough (e.g., plant seeds, Drosophila embryos), in the entire tissue (whole mount ISH), in cells, and in circulating tumour cells (CTCs). This is distinct from immunohistochemistry, which usually localizes proteins in tissue sections.

In situ hybridization is a powerful technique for identifying specific mRNA species within individual cells in tissue sections, providing insights into physiological processes and disease pathogenesis. However, in situ hybridization requires that many steps be taken with precise optimization for each tissue examined and for each probe used. In order to preserve the target mRNA within tissues, it is often required that crosslinking fixatives (such as formaldehyde) be used.

Degradation of target RNA is a problem in ISH experiments. The methods of the invention provide a solution to this problem by targeting stable regions within target RNA of interest.

### Microarray

A DNA microarray (also commonly known as DNA chip or biochip) is a collection of microscopic DNA spots attached to a solid surface. Scientists use DNA microarrays to measure the expression levels of large numbers of genes simultaneously or to genotype multiple regions of a genome. Each DNA spot contains picomoles (10-12 moles) of a specific DNA sequence, known as probes (or reporters or oligos). These can be a short section of a gene or other DNA element that are used to hybridize a cDNA or cRNA (also called anti-sense RNA) sample (called target) under high-stringency conditions. Probe-target hybridization is usually detected and quantified by detection of fluorophore-, silver-, or chemiluminescence-labeled targets to determine relative abundance of nucleic acid sequences in the target.

The present invention has application for microarray analysis of tissues, including tissues that are subject to degradation. By designing probes, to include on the microarray chip, that target stable regions of RNA (according to the present invention), the microarray analysis may provide a more realistic representation of the in vivo expression profile, that is not so skewed by degradation after RNA is extracted from the tissue sample. Such chips would also be able to be used to screen samples containing RNA, including degraded RNA, in order to type the source of that RNA as has been previously described.

### NanoString nCounter

NanoString's nCounter technology is a variation on the DNA microarray and was invented and patented by Krassen Dimitrov and Dwayne Dunaway. It uses molecular “barcodes” and microscopic imaging to detect and count up to several hundred unique RNAs in one hybridization reaction. Each color-coded barcode is attached to a single target-specific probe corresponding to a gene of interest.

The NanoString protocol includes the following steps:
Hybridization: NanoString's Technology employs two ˜50 base probes per mRNA that hybridize in solution. The reporter probe carries the signal, while the capture probe allows the complex to be immobilized for data collection.
Purification and Immobilization: After hybridization, the excess probes are removed and the probe/target complexes are aligned and immobilized in the nCounter Cartridge.
Data Collection: Sample Cartridges are placed in the Digital Analyzer instrument for data collection. Color codes on the surface of the cartridge are counted and tabulated for each target molecule.

The nCounter Analysis System: The system consists of two instruments: the Prep Station, which is an automated fluidic instrument that immobilizes CodeSet complexes for data collection, and the Digital Analyzer, which derives data by counting fluorescent barcodes. As the NanoString nCounter system is dependent on probe-target hybridisation for RNA detection and analysis, this invention has immediate application to NanoString nCounter. NanoString nCounter probe design (target hybridisation sites) are designed to conform to certain thermodynamic requirements and gives no consideration to target RNA degradation or stability. Therefore we believe that with this invention NanoString nCounter RNA detection can be vastly improved by designing probes to hybridise to stable regions in the RNA sequence.

### Samples

The sample may be any type of biological sample that includes RNA.

Samples suitable for in situ hybridisation include biological tissue sections.

Preferably the forensic sample is selected from the group comprising blood, semen (with or without spermatazoa), and menstrual fluid.

### RNA Extraction

RNA extraction procedures are well known to those skilled in the art. Examples include: Acid guanidium thiocyanate-phenol-chloroform RNA extraction (Chomczynski, Piotr, and Nicoletta Sacchi. The single-step method of RNA isolation by acid guanidinium thiocyanate-phenol-chloroform extraction: twenty-something years on. Nature protocols 1(2) (2006): 581-585); magnetic bead-based RNA extraction (Berensmeier, Sonja. “Magnetic particles for the separation and purification of nucleic acids.” Applied microbiology and biotechnology 73(3) (2006): 495-504); column-based RNA purification (Matson, R. S. (2008). Microarray Methods and Protocols. Boca Raton, Fla.: CRC. pp. 7-29. ISBN 1420046659; Kumar, A. (2006). Genetic Engineering. New York: Nova Science Publishers. pp. 101-102. ISBN 159454753X); and TRIzol (TRI reagent) RNA extraction (Rio, D. C., Ares, M., Hannon, G. J., & Nilsen, T. W. Purification of RNA using TRIzol (TRI reagent). Cold Spring Harbor Protocols, (2010), pdb-prot5439).

### RNA Sequencing and Stable Region Identification

RNA sequencing refers to sequencing of all RNA in a sample using what is commonly known as Next Generation Sequencing (NGS) (second generation sequencing or massively parallel sequencing; Mardis, E. R. (2008). The impact of next-generation sequencing technology on genetics. Trends in genetics, 24(3), 133-141; Metzker, M. L. (2010). Sequencing technologies—the next generation. Nature Reviews Genetics, 11(1), 31-46; Reis-Filho, J. S. (2009). Next-generation sequencing. Breast Cancer Res, 11(Suppl 3), S12 and Schuster, S. C. (2008). Next-generation sequencing transforms today's biology. Nature methods, 5(1), 16-18). Although different sequencing instrumentation manufacturers employ slightly different sequencing chemistry, RNA sequencing can be achieved using any of these NGS (massively parallel sequencing) technologies (Mardis, 2008 and Mutz, K. O., Heilkenbrinker, A., Lönne, M., Walter, J. G., & Stahl, F. (2013). Transcriptome analysis using next-generation sequencing. Current opinion in biotechnology, 24(1), 22-30). As there are many NGS technologies available, there are small differences in the methodology for RNA sequencing. The following is a description of how RNA sequencing using NGS works in general (Metzker, 2010):
Total RNA is extracted from the sample of interest, using a common RNA extraction method. Post-extraction processes can be used to enrich the RNA sample.
Complimentary DNA (cDNA) is then synthesised using extracted RNA. cDNA is then used as the template for RNA sequencing.
NGS uses variations of sequencing by synthesis (SBS) chemistry (Fuller, C. W.,

Middendorf, L. R., Benner, S. A., Church, G. M., Harris, T., Huang, X., . . . & Vezenov, D. V. (2009). The challenges of sequencing by synthesis. Nature biotechnology, 27(11), 1013-1023). With cDNA as a template, new nucleotide fragments, known as reads, are synthesised base by base, with each incorporated base recorded during sequencing (Fuller, 2009).
The data output from RNA sequencing is a list of all the reads generated, and their sequence (Fuller, 2009 and Metzker, 2010). This data undergoes quality assessment (Patel, R. K., & Jain, M. (2012). NGS QC Toolkit: a toolkit for quality control of next generation sequencing data. PloS one, 7(2), e30619). For RNA sequencing, sequencing reads are then aligned to the reference genome using a splice-aware sequence alignment algorithm (Trapnell, C., Pachter, L., & Salzberg, S. L. (2009). TopHat: discovering splice junctions with RNA-Seq. Bioinformatics, 25(9), 1105-1111).

Alignments can then be visualised using any genome browser or sequence viewing software. RNA stable regions are identified by viewing sequencing read alignments along the RNA of interest. Regions along the RNA sequence where there more reads aligned (high read coverage) are deemed to be stable regions.

### Stable Regions

A stable region of an RNA sequence according to the invention is a region within any given RNA sequence that RNA sequencing data shows produces more aligned sequencing reads than at least one other region with the same RNA sequence.

In a preferred embodiment the stable region has at least 1.1× more preferably 1.2×, more preferably 1.3×, more preferably 1.4×, more preferably 1.5×, more preferably 1.6×, more preferably 1.7×, more preferably 1.8×, more preferably 1.9×, more preferably 2.0×, more preferably 2.2×, more preferably 2.4×, more preferably 2.6×, more preferably 2.8×, more preferably 3.0×, more preferably, 3.2×, more preferably 3.4×, more preferably 3.6×, more preferably 3.8×, more preferably 4.0×, more preferably 4.2×, more preferably 4.4×, more preferably 4.6×, more preferably 4.8×, more preferably 5.0× as many aligned reads than at least one other region within the same RNA sequence.

### PCR-Based Methods

PCR-based methods are particularly preferred for detection of RNA sequence in the method of the invention.

General PCR approaches are well known to those skilled in the art (Mullis et al., 1994). Various other developments of the basic PCR approach may also be advantageous applied to the method of the invention. Examples are discussed briefly below.

### Multiplex-PCR

Multiplex-PCR utilises multiple primer sets within a single PCR reaction to produce amplified products (amplicons) of varying sizes that are specific to different target RNA, cDNA or DNA sequences. By targeting multiple sequences at once, diagnostic information may be gained from a single reaction that otherwise would require several times the reagents and more time to perform. Annealing temperatures and primer sets are generally optimized to work within a single reaction, and produce different amplicon sizes. That is, the amplicons should form distinct bands when visualized by gel electrophoresis. Multiplex PCR can be used in the method of the invention to distinguish the type of sample it applied to in a single sample or reaction.

### MLPA

Multiplex ligation-dependent probe amplification (MLPA) (U.S. Pat. No. 6,955,901) is a variation of the multiplex polymerase chain reaction that permits multiple targets to be amplified with only a single primer pair. Each probe consists of two oligonucleotides which recognise adjacent target sites on the DNA. One probe oligonucleotide contains the sequence recognised by the forward primer, the other the sequence recognised by the reverse primer. Only when both probe oligonucleotides are hybridised to their respective targets, can they be ligated into a complete probe. The advantage of splitting the probe into two parts is that only the ligated oligonucleotides, but not the unbound probe oligonucleotides, are amplified. If the probes were not split in this way, the primer sequences at either end would cause the probes to be amplified regardless of their hybridization to the template DNA. Each complete probe has a unique length, so that its resulting amplicons can be separated and identified (for example by capillary electrophoresis among other methods). Since the forward primer used for probe amplification is fluorescently labeled, each amplicon generates a fluorescent peak which can be detected by a capillary sequencer. Comparing the peak pattern obtained on a given sample with that obtained on various reference samples measures presence or absence (or the relative quantity) of each amplicon can be determined. This then indicates presence or absence (or the relative quantity) of the target sequence is present in the sample DNA. The products can also be detected using gel electrophoresis or microfluidic systems such as Shimadzu MultiNA. The use of reference samples to establish presence or absence is the same. More information about MLPA is available on the World Wide Web at http://www.mlpa.com. MLPA probes may be synthesized as oligonucleotides, by methods known to those skilled in the art. MLPA probes and reagents may be commercially produced by and purchased from HRC-Holland (http://www.mIpa.com).

### Quantitative PCR

Quantitative PCR (Q-PCR) is used to measure the quantity of a PCR product (commonly in real-time). Q-PCR quantitatively measures starting amounts of DNA, cDNA, or RNA. Q-PCR is commonly used to determine whether a DNA sequence is present in a sample and the number of its copies in the sample. Quantitative real-time PCR has a very high degree of precision. Q-PCR methods use fluorescent dyes, such as SYBR Green, EvaGreen or fluorophore-containing DNA probes, such as TaqMan, to measure the amount of amplified product in real time. Q-PCR is sometimes abbreviated to RT-PCR (Real Time PCR) or RQ-PCR. QRT-PCR or RTQ-PCR.

### Primers

The term “primer” refers to a short polynucleotide, usually having a free 3′OH group, that is hybridized to a template and used for priming polymerization of a polynucleotide complementary to the template. Such a primer is preferably at least 5, more preferably at least 6, more preferably at least 7, more preferably at least 9, more preferably at least 10, more preferably at least 11, more preferably at least 12, more preferably at least 13, more preferably at least 14, more preferably at least 15, more preferably at least 16, more preferably at least 17, more preferably at least 18, more preferably at least 19, more preferably at least 20 nucleotides in length.

In conventional primer design for amplifying RNA marker sequences, primers are typically designed to cover exon boundaries, to prevent amplification of genomic DNA.

The invention relates to targeting stable regions of RNA transcripts, which is particularly useful when amplifying markers from degraded samples. As will be readily apparent, once a stable region is identified, that region can be used to type samples containing RNA having RIN values from 8 to 10 as well as below 8. Both options thus form part of the present invention.

In one embodiment the primer of the invention for use a method of the invention, does not span an exon boundary.

Although not preferred, in one embodiment the primer of the invention for use a method of the invention, may span an exon boundary.

### Labelling of Primers

Methods for labelling primers are well known to those skilled in the art, and include: Primers can be labelled enzymatically (Davies, M. J., Shah, A., & Bruce, I. J. (2000). Synthesis of fluorescently labelled oligonucleotides and nucleic acids. Chemical Society Reviews, 29(2), 97-107.) or chemically (including automated solid-phase chemical synthesis) (Proudnikov, D., & Mirzabekov, A. (1996). Chemical methods of DNA and RNA fluorescent labeling. Nucleic acids research, 24(22), 4535-4542.).

Primers can be labelled with; a fluorescence label (fluorophore, Kutyavin, I. V., Afonina, I. A., Mills, A., Gorn, V. V., Lukhtanov, E. A., Belousov, E. S., ... & Hedgpeth, J. (2000). 3′-minor groove binder-DNA probes increase sequence specificity at PCR extension temperatures. Nucleic Acids Research, 28(2), 655-661.)), biotin (Pon, R. T. (1991). A long chain biotin phosphoramidite reagent for the automated synthesis of 5′-biotinylated oligonucleotides. Tetrahedron letters, 32(14), 1715-1718.), or radioactive and non-radioactive labels (for example digoxigenin) (Agrawal, S., Christodoulou, C., & Gait, M. J. (1986). Efficient methods for attaching non-radioactive labels to the 5′ ends of synthetic oligodeoxyribonucleotides. Nucleic acids research, 14(15), 6227-6245.).

Primers labelled by such methods form part of the invention.

### Probe-Based Methods

Probe-based methods may be applied to detect the RNA sequences in the method of the invention. Methods for hybridizing probes to target nucleic acid sequences are well known to those skilled in the art (Sambrook et al., Eds, 1987, Molecular Cloning, A Laboratory Manual, 2nd Ed. Cold Spring Harbor Press).

Probe-based methods include in situ hybridization.

The term “probe” refers to a short polynucleotide that is used to detect a polynucleotide sequence that is at least partially complementary to the probe, in a hybridization-based assay. The probe may consist of a “fragment” of a polynucleotide as defined herein. Preferably such a probe is at least 10, more preferably at least 20, more preferably at least 30, more preferably at least 40, more preferably at least 50, more preferably at least 100, more preferably at least 200, more preferably at least 300, more preferably at least 400 and most preferably at least 500 nucleotides in length.

### Labelling of Probes

Methods for labelling probes are well known to those skilled in the art, and include: Probes can be labelled enzymatically (Sambrook, et al. 1987; Davies, et al., 2000) or chemically (including automated solid-phase chemical synthesis) (Proudnikov, et al. 1996).

Probes can be:

Molecular Beacon (Tyagi, S., & Kramer, F. R. (1996). Molecular beacons: probes that fluoresce upon hybridization. Nature biotechnology, (14), 303-8.),

TaqMan (Kutyavin I V, Afonina I A, Mills A, Gorn V V, Lukhtanov E A, Belousov E S, Singer M J, Walburger D K, Lokhov S G, Gall A A, Dempcy R, Reed M W, Meyer R B, Hedgpeth J (2000). 3′-minor groove binder-DNA probes increase sequence specificity at PCR extension temperatures. Nucleic Acids Research, 28(2), 655-661.

Scorpion (R Carters, R., Ferguson, J., Gaut, R., Ravetto, P., Thelwell, N., & Whitcombe, D. (2008). Design and use of scorpions fluorescent signaling molecules. In Molecular beacons: Signalling nucleic acid probes, methods, and protocols (pp. 99-115). Humana Press.

In situ hybridization probes- Eisel, D.; Grunewald-Janho, S.; Krushen, B., ed. (2002). DIG Application Manual for Nonradioactive in situ Hybridization (3rd ed.). Penzberg: Roche Diagnostics.

Radioactive and non-radioactive (Simmons, D. M., Arriza, J. L., & Swanson, L. W. (1989). A complete protocol for in situ hybridization of messenger RNAs in brain and other tissues with radio-labeled single-stranded RNA probes. Journal of Histotechnology, 12(3), 169-181; Agrawal, S., Christodoulou, C., & Gait, M. J. (1986). Efficient methods for attaching non-radioactive labels to the 5′ ends of synthetic oligodeoxyribonucleotides. Nucleic acids research, 14(15), 6227-6245.).

Probes labelled by such methods form part of the invention.

### Polynucleotides

The term “polynucleotide(s),” as used herein, means a single or double-stranded deoxyribonucleotide or ribonucleotide polymer of any length but preferably at least 5 nucleotides, and include as non-limiting examples, coding and non-coding sequences of a gene, sense and antisense sequences complements, exons, introns, genomic DNA, cDNA, pre-mRNA, mRNA, rRNA, siRNA, miRNA, tRNA, naturally occurring DNA or RNA sequences, synthetic RNA and DNA sequences, and fragments thereof. In one embodiment the nucleic acid is isolated, that is separated from its normal cellular environment. The term “nucleic acid” can be used interchangeably with “polynucleotide”.

### Methods for Extracting Nucleic Acids

Methods for extracting nucleic acids are well-known to those skilled in the art (Sambrook et al., Eds, 1987, Molecular Cloning, A Laboratory Manual, 2nd Ed. Cold Spring Harbor Press).

Specialised extraction procedures can optionally be applied depending on the sample type, as discussed in the example section. For example, RNA from forensic type samples can be extracted using a DNA-RNA co-extraction method, as described by Bowden et al. 2011 (Bowden, A., Fleming, R., & Harbison, S. (2011). A method for DNA and RNA co-extraction for use on forensic samples using the Promega DNA IQ™ system. Forensic Science International: Genetics, 5(1), 64-68).

All such methods are intended to be included within the scope of the present invention.

### Percent Identity

Variant polynucleotide sequences preferably exhibit at least 70%, more preferably at least 71%, more preferably at least 72%, more preferably at least 73%, more preferably at least 74%, more preferably at least 75%, more preferably at least 76%, more preferably at least 77%, more preferably at least 78%, more preferably at least 79%, more preferably at least 80%, more preferably at least 81%, more preferably at least 82%, more preferably at least 83%, more preferably at least 84%, more preferably at least 85%, more preferably at least 86%, more preferably at least 87%, more preferably at least 88%, more preferably at least 89%, more preferably at least 90%, more preferably at least 91%, more preferably at least 92%, more preferably at least 93%, more preferably at least 94%, more preferably at least 95%, more preferably at least 96%, more preferably at least 97%, more preferably at least 98%, and most preferably at least 99% identity to a specified polynucleotide sequence. Identity is found over a comparison window of at least 10 nucleotide positions, more preferably at least 10 nucleotide positions, more preferably at least 12 nucleotide positions, more preferably at least 13 nucleotide positions, more preferably at least 14 nucleotide positions, more preferably at least 15 nucleotide positions, more preferably at least 16 nucleotide positions, more preferably at least 17 nucleotide positions, more preferably at least 18 nucleotide positions, more preferably at least 19 nucleotide positions, more preferably at least 20 nucleotide positions, more preferably at least 21 nucleotide positions and most preferably over the entire length of the specified polynucleotide sequence. The invention includes such variants.

Polynucleotide sequence identity can be determined in the following manner. The subject polynucleotide sequence is compared to a candidate polynucleotide sequence using BLASTN (from the BLAST suite of programs, version 2.2.5 [Nov 2002]) in bl2seq (Tatiana A. Tatusova, Thomas L. Madden (1999), “Blast 2 sequences—a new tool for comparing protein and nucleotide sequences”, FEMS Microbiol Lett. 174:247-250), which is publicly available from NCBI (ftp://ftp.ncbi.nih.gov/blast/). The default parameters of bl2seq are utilized except that filtering of low complexity parts should be turned off.

The identity of polynucleotide sequences may be examined using the following unix command line parameters:

bl2seq -i nucleotideseq1 -j nucleotideseq2 -F F -p blastn

The parameter -F F turns off filtering of low complexity sections. The parameter -p selects the appropriate algorithm for the pair of sequences. The bl2seq program reports sequence identity as both the number and percentage of identical nucleotides in a line “Identities=”.

Polynucleotide sequence identity may also be calculated over the entire length of the overlap between a candidate and subject polynucleotide sequences using global sequence alignment programs (e.g. Needleman, S. B. and Wunsch, C. D. (1970) J. Mol. Biol. 48, 443-453). A full implementation of the Needleman-Wunsch global alignment algorithm is found in the needle program in the EMBOSS package (Rice, P. Longden, I. and Bleasby, A. EMBOSS: The European Molecular Biology Open Software Suite, Trends in Genetics June 2000, vol 16, No 6. pp.276-277) which can be obtained from http://www.hgmp.mrc.ac.uk/Software/EMBOSS/. The European Bioinformatics Institute server also provides the facility to perform EMBOSS-needle global alignments between two sequences on line at http:/www.ebi.ac.uk/emboss/align/.

Alternatively the GAP program, which computes an optimal global alignment of two sequences without penalizing terminal gaps, may be used to calculate sequence identity. GAP is described in the following paper: Huang, X. (1994) On Global Sequence Alignment. Computer Applications in the Biosciences 10, 227-235.

Sequence identity may also be calculated by aligning sequences to be compared using Vector NTI version 9.0, which uses a Clustal W algorithm (Thompson et al., 1994, Nucleic Acids Research 24, 4876-4882), then calculating the percentage sequence identity between the aligned sequences using Vector NTI version 9.0 (Sep. 2, 2003 ©1994-2003 InforMax, licensed to Invitrogen).

In general terms therefore the invention provides a method for the detection of an RNA sequence in a sample. The method including the steps of:
a) providing a sample, and
b) detecting the RNA sequence using at least one primer or probe complementary to a stable region of the RNA sequence.

The stable region of the RNA sequence will preferably be identified using RNA sequencing of the sample and, in particular, will be identified as a region in the RNA sequence which has more aligned sequencing reads than another region, or regions, of the same RNA sequence.

Stable regions have been identified and discussed herein and stable regions for use in the methods of the invention can be selected from the group comprising SEQ ID NO:1 to SEQ ID NO:95 or a compliment of anyone thereof.

Primers have also been identified and discussed herein and primers can be selected from the group comprising SEQ ID NO:96 to SEQ ID NO:107 or compliment of anyone thereof.

Additionally, in a more specific sense, the invention can be seen to include a nucleotide sequence comprising at least 5 nucleotides with at least 70% identity to a sequence selected from SEQ ID NO:1 to SEQ ID NO:95 or a compliment thereof.

Further, and again in a more specific sense, the invention can be seen to include a nucleotide sequence comprising at least 5 nucleotides of a sequence selected from SEQ ID NO:1 to SEQ ID NO:95 or a compliment thereof.

Further, and again in a more specific sense, the invention can be seen to include a nucleotide sequence comprising at least 10 nucleotides with at least 70% identity to a sequence selected from SEQ ID NO:1 to SEQ ID NO:95 or a compliment thereof.

Further, and again in a more specific sense, the invention can be seen to include a nucleotide sequence comprising at least 10 nucleotides of a sequence selected from SEQ ID NO:1 to SEQ ID NO:95 or a compliment thereof.

Further, and again in a more specific sense, the invention to be seen to include a nucleotide sequence selected from any one of SEQ ID NO:96 to SEQ ID NO:107

The use of a nucleotide sequence as is defined above in the typing of a sample including RNA specifically forms part of the present invention.

As will be apparent, samples containing RNA can be taken from a variety of sources. The most preferable sample is a biological tissue sample which can be either solid or liquid.

The method of the present invention is particularly suitable for use in the forensic field and therefore the sample can be a forensic sample of any type containing RNA such as selected from the group comprising blood, semen (with or without spermatozoa), and menstrual fluid.

The RNA should preferably be extracted from the sample prior to the detecting step and the RNA sequence can be detected directly or indirectly as will be known to a skilled person. It is however referred that the RNA sequence is detected indirectly by detection of a complementary DNA (cDNA) corresponding to the RNA sequence.

The invention, in a more particular sense, can also be seen to include a method of typing a sample including RNA where the method includes the steps of:
a) providing a sample including RNA;
b) detecting one or more stable RNA sequences in the sample using at least one primer or probe complementary to the one or more stable region of the RNA;

wherein the stable RNA sequence is specific for the type of sample; and

wherein detecting the stable RNA sequence indicates the type of sample.

The invention, in another sense, can be seen to include a method of typing a sample including degraded RNA, the method including the steps:
a) providing a sample including degraded RNA;
b) detecting one or more stable RNA sequences in the sample using at least one primer or probe complementary to the one or more stable region of the degraded RNA;

wherein the stable RNA sequence is specific for the type of sample; and

wherein detecting the target RNA sequence indicates the type of sample.

In another embodiment the invention can be a method for the identification of a stable region in RNA in a sample, the method comprising:
a) providing a sample including RNA,
b) isolating total RNA from the sample,
c) removing DNA from the sample
d) generating cDNA complementary to the RNA in the sample,
e) sequencing the cDNA.

wherein the stable region of the RNA sequence is identified as a region in the RNA sequence which has more aligned sequencing reads than another region, or regions, of the same RNA sequence.

As has been previously discussed, the method can be applied to RNA which has degraded to a condition which had previously been thought not to be useful as a means for typing/identifying the source of the sample from which it has been extracted. The methods of the invention can be used to type/identify the source of samples in which the RNA content has a RIN value of less than 8. As stable regions in RNA having a value of less than eight will also be present in RNA having a RIN value of between 8 and 10, once the stable regions have been identified those stable regions can also be used to identify/type the source of the sample having an RIN of between 8 and 10. Therefore, the method can be used to type/identify the source of samples having any RIN value, including samples in which the RIN value cannot be determined.

As has been discussed previously, the stable region of the RNA sequence can be identified as a region in the RNA sequence which has more aligned sequencing reads than another region, or regions, of the same RNA sequence.

As will be readily apparent to a skilled person, the RNA sequence will preferably be detected using a primer or a probe. As will also be apparent, the RNA sequence can be detected using more than one primer or probe (e.g. two primers) if appropriate/desired.

The primers and should preferably correspond to, or be complementary to, or be capable of hybridising to, a sequence within the stable region of the RNA that has been extracted from the sample. The primers are used to amplify the part of the stable region bound by the primers, such as by a polymerase chain reaction (PCR) method. The PCR method can be selected from standard PCR, reverse transcriptase (RT)-PCR, and quantitative reverse transcriptase PCR (qRT-PCR).

In addition, and as will also be readily apparent to a skilled person, the RNA sequence can be detected using a probe. This will preferably correspond to, or be complementary to, a sequence within the stable region of the RNA that has been extracted from the sample.

The RNA sequence can be encoded by a marker gene specific for the type of sample. That is, the expression of the RNA sequence, or presence of the RNA sequence, in the sample, is diagnostic for the type of sample. For example, when the sample is circulatory blood, the marker gene is selected from:
Hemoglobin delta (HBD),
Solute carrier family 4 (anion exchanger), member 1 (Diego blood group) (SLC4A1).

When sample contains spermatazoa, the marker gene is Transition protein 1 (during histone to protamine replacement) (TNP1).

When the sample is seminal fluid, the marker gene is Kallikrein-related peptidase 2 (KLK2).

When the sample is menstrual fluid, the marker gene is selected from:
Matrix metallopeptidase 3 (MMP3), and
Stanniocalcin 1 (STC1).

The detection process of the present invention can involve the use of either a primer or a probe capable of hybridising to the stable region of the RNA sequence, or a cDNA corresponding to the stable region or a complement thereof. The method may involve using just one pair of primers, or a single probe, to type the sample. Alternatively multiple pairs of primers, or multiple probes, may be used.

The primer or the probe can include (i) a sequence of at least 5 nucleotides with at least 70% identity to any part of the sequence of any one of SEQ ID NO:1 to 95 or a complement thereof or (ii) a sequence of at least 5 nucleotides with at least 70% identity to the sequence of any one of SEQ ID NO:1 to 95, or a complement thereof or (iii) a sequence of at least 5 nucleotides of the sequence of any one of SEQ ID NO:1 to 95, or a complement thereof or (iv) a sequence of at least 5 nucleotides of the sequence of any one of SEQ ID NO:1 to 95, or a complement thereof or (v) a sequence selected from any one of SEQ ID NO:96 to 107 or (vi) a label or tag attached to a sequence selected from any one of those sequences.

The primer or the probe can include (i) a sequence of at least 10 nucleotides with at least 70% identity to any part of the sequence of any one of SEQ ID NO:1 to 95 or a complement thereof or (ii) a sequence of at least 10 nucleotides with at least 70% identity to the sequence of any one of SEQ ID NO:1 to 95, or a complement thereof or (iii) a sequence of at least 10 nucleotides of the sequence of any one of SEQ ID NO:1 to 95, or a complement thereof or (iv) a sequence of at least 10 nucleotides of the sequence of any one of SEQ ID NO:1 to 95, or a complement thereof or (v) a sequence selected from any one of SEQ ID NO:96 to 107 or (vi) a label or tag attached to a sequence selected from any one of those sequences.

By way of example, typing of a sample can be undertaken using multiplex PCR performed with multiple primers, at least one of which is diagnostic for the type of sample.

Preferably multiplex PCR is performed using at least 4, more preferably at least 5, more preferably at least 6, more preferably at least 7, more preferably at least 8, more preferably at least 9, more preferably at least 10, more preferably at least 11, more preferably at least 12, more preferably at least 13, more preferably at least 14, more preferably at least 15, more preferably at least 16, more preferably at least 17, more preferably at least 18, more preferably at least 19, more preferably at least 20, more preferably at least 21, more preferably at least 22, more preferably at least 23, more preferably at least 24, more preferably at least 25, more preferably at least 26, more preferably at least 27, more preferably at least 28, more preferably at least 29, more preferably at least 30 primers of the invention.

The invention also allows the provision of a kit that includes at least one primer or probe according to the present invention. Such a kit can include any number of primers or probes and in particular the kit can include at least 2, more preferably at least 3, more preferably at least 4, more preferably at least 5, more preferably at least 6, more preferably at least 7, more preferably at least 8, more preferably at least 9, more preferably at least 10, more preferably at least 11, more preferably at least 12, more preferably at least 13, more preferably at least 14, more preferably at least 15, more preferably at least 16, more preferably at least 17, more preferably at least 18, more preferably at least 19, more preferably at least 20, more preferably at least 21, more preferably at least 22, more preferably at least 23, more preferably at least 24, more preferably at least 25, more preferably at least 26, more preferably at least 27, more preferably at least 28, more preferably at least 29, more preferably at least 30 primers or probes of the invention. Combinations of primers and probes may also be provided in such kits.

As will be readily apparent, the kit should also include instructions for use, if such instructions are needed.

The invention also allows the provision of microarrays or chips or like products that include sequences that have been identified herein as stable areas of RNA that can be used to type/identify samples or that are complimentary thereto. These sequences have been used to generate primers and probes that can be used on microarrays or chips or like products for the detection of nucleotide sequences.

Such microarrays or chips are of particular commercial importance as they allow the efficient and accurate identification of unknown samples including RNA, including where the RNA has been degraded. The creation of such products as well within the abilities of the person skilled in the art once they have the benefit of knowledge of the present invention.

### BRIEF DESCRIPTION OF DRAWINGS

FIG. 1. Expression patterns of HBD, SLC4A1, TNP1, KLK2, MMP3 and STC1. Amplification of six samples per body fluid; BL=circulatory blood, SA=saliva/buccal, SM=semen (with spermatozoa), SF=seminal fluid (without spermatozoa), MF=menstrual fluid, VM=vaginal material. The same samples and donors were not necessarily used for the assessment of all markers.

FIG. 2. Sensitivity comparison of the six novel mRNAs to four well-known markers [1]. Top: HBD and SLC4A1 compared to GYPA using three samples each of 2, 1 and 0.5 pL circulatory blood and a primer concentration of 0.2 μM. Second from top: TNP1 compared to PRM2 using 9 samples of 1 μL semen from three donors and a primer concentration of 0.05 μM. Second from bottom: KLK2 compared to TGM4 using three samples each of 2, 1 and 0.5 μL seminal fluid (azoospermic) and a primer concentration of 0.1 μM. Bottom: MMP3 and STC1 compared to MMP11 using nine menstrual fluid samples (days 2 and 3) from two donors and a primer concentration of 0.1 μM. Average peak heights (APH) and standard deviations were calculated from three technical replicates.

The invention will now be exemplified by way of the following non-limiting examples.

### EXAMPLE 1

### Identification of RNA Stable Regions in Body Samples

### Materials and Methods

### Identification of Body Fluid-Specific Candidate Genes

Candidate mRNAs for the identification of circulatory blood (HBD, SLC4A1) and menstrual fluid (MMP3, STC1) were selected from RNA-Seq data of degraded body fluids as published previously [31]. Semen marker candidates (TNP1, KLK2) were chosen from gene expression databases (TIGER, PaGenBase) [32,33] with respect to their physiological function in the body.

### Primer Design

Primers for HBD, SLC4A1, MMP3 and STC1 were designed to target transcript stable regions (StaRs) as described previously [34] using the OligoAnalyzer 3.1 online tool (Integrated DNA Technologies, Inc., Coralville, Iowa, USA). Sequencing coverage maps were viewed using the Geneious v.5.6.7 software (Biomatters Ltd., Auckland, New Zealand) and regions of high coverage selected for primer design. Primers for TNP1 and KLK2 were designed using conventional primer design strategy. The specificity of all primers to their intended mRNA targets was verified using Primer-BLAST [35]. Primer sequences and expected amplicon sizes are listed in Table 1.

TABLE 1
Primer sequences and expected amplicon
sizes of the novel body fluid markers.

| Target | | Accession | | Product size |
|---|---|---|---|---|
| body fluid | Marker | number | Primer Sequence (5′-3′) | (bp) |
| | Haemoglobin | NM_000519.3 | F: ACTGCTGTCAATGCCCTGTG | 176 |
| | delta (HBD) | | R: ACCTTCTTGCCATGAGCCTT | |
| Circulatory | Solute carrier | NM_000342.3 | F: AACTGGACACTCAGGACCAC | 102 |
| blood | family 4 (anion | | R: GGATGTCTGGGTCTTCATATTCCT | |
| | exchanger), | | | |
| | member 1, (Diego | | | |
| | blood group) | | | |
| | (SLC4A1) | | | |
| Semen | Transition protein | NM_003284.3 | F: GATGACGCCAATCGCAATTACC | 102 |
| containing | 1 (during histone | | R: CCTTCTGCTGTTCTTGTTGCTG | |
| spermatozoa | to protamine | | | |
| | replacement (TNP1) | | | |
| Seminal | Kallikrein-related | NM_005551.4 | F: CAGTCATGGATGGGCACACT | 141 |
| fluid | peptidase 2 | | R: ACCCTCTGGCCTGTGTCTTC | |
| | (KLK2) | | | |
| | Matrix | NM_002422.3 | F: CCATGCCTATGCCCCTG | 84 |
| | metallopeptidase 3 | | R: GTCCCTGTTGTATCCTTTGTCC | |
| | (MMP3) | | | |
| Menstral | Stanniocalcin 1 | NM_003155.2 | F: TGCCCAATCACTTCTCCAACAG | 103 |
| fluid | (STC1) | | R: TTCTCCATCAGGCTGTCTCTG | |

### Collection of Body Fluid Samples

Six samples each of 50 μL circulatory blood, semen and seminal fluid (azoospermic), as well as saliva/buccal mucosa, menstrual and non-menstrual vaginal swabs were obtained from healthy, consenting volunteers, as approved by the University of Auckland Human Participants Ethics Committee (UAHPEC). Blood was drawn using a sterile AKKU-CHEK® Safe-T-Pro Plus lancet (Roche Diagnostics USA, Indianapolis, Ind., USA). Blood, semen and seminal fluid aliquots were deposited onto sterile Cultiplast® rayon swabs. Buccal, menstrual and vaginal samples were obtained by volunteers themselves using sterile swabs. All samples were allowed to dry overnight at ambient laboratory conditions and then extracted as described below.

### RNA Extraction and Purification

Total RNA from body fluid samples was prepared as described previously [31,34] using the Promega® DNA IQ and ReliaPrep™ RNA Cell Miniprep Systems (Promega Corporation, Madison, Wis., USA) following the manufacturer's instructions. Genomic DNA was removed by incorporating an on-column DNase I treatment during the RNA extraction process. RNA was eluted in 45 μL nuclease-free water. The absence of genomic DNA was verified by real-time PCR using the Quantifiler® Human DNA quantification kit (Life Technologies™ by Thermo Fisher Scientific, Inc., Waltham, Mass., USA) with 1 μL purified RNA in a 12.5 μL reaction. Samples which contained residual DNA were treated with TURBO™ DNase (Invitrogen™ by Thermo Fisher Scientific, Inc.) and re-quantified until no DNA was detectable.

### cDNA Synthesis

Complementary DNA (cDNA) was prepared using the High Capacity cDNA Reverse Transcription Kit (Applied Biosystems™ by Thermo Fisher Scientific, Inc.) according to the manufacturer's instructions. Ten microlitres of DNA-free RNA were subjected to reverse transcription in a 20 μL reaction. Synthesis was performed on a GeneAmp PCR System 9700 thermal cycler (Applied Biosystems™ by Thermo Fisher Scientific, Inc.) using the following program: 25° C. for 10 min, 37° C. for 120 min, followed by 85° C. for 5 min and hold at 4° C.

### Polymerase Chain Reaction (PCR)

### PCR Reactions

Body fluid cDNA samples were amplified using the QIAGEN® Multiplex PCR Kit (Qiagen GmbH, Hilden, Germany) according to the manufacturer's instructions. Two microlitres of cDNA were amplified in 25 μL PCR reactions containing 12.5 μL of 2×PCR master mix. Primer concentrations for specificity testing were as follows: 0.05 μM (HBD), 0.03 μM (SLC4A1), 0.08 μM (TNP1), 0.4 μM (KLK2), 0.02 μM (MMP3), 0.02 μM (STC1). Primer concentrations for comparison were 0.2 μM (circulatory blood), 0.05 μM (semen), and 0.1 μM (seminal and menstrual fluid), respectively. Finally, nuclease-free water was added to achieve a total volume of 25 μL for each reaction.

### PCR Cycling Conditions

PCR cycling conditions for amplification on the GeneAmp PCR System 9700 were as published previously [31,34,36]: initial denaturation at 95° C. for 15 min, followed by 35 cycles of 94° C. for 30 s, 58° C. for 3 min and 72° C. for 1 min, final elongation at 72° C. for 45 min and cooling down to 4° C.

### Capillary Electrophoresis and Data Analysis

PCR products were separated on a Genetic Analyzer 3130×l (Applied Biosystems™ by Thermo Fisher Scientific, Inc.). One microlitre of amplified PCR product was mixed with 9 μL of a formamide/size standard stock solution, created by adding 15 μL GeneScan™ 500 ROX™ to 1000 μL HiDi™ formamide. Results were analysed with GeneMapper v.3.2.1 (Applied Biosystems™ by Thermo Fisher Scientific, Inc.).

### Results and Discussion

### Selection of Body Fluid Marker Candidates

Whole transcriptome paired-end sequencing (2×100 bp) of circulatory blood (2 donors) and menstrual fluid (1 donor) was performed in order to identify highly expressed biomarkers possibly exclusive to each body fluid type [31]. Processed and merged sequencing reads for each sample were aligned to the human reference sequence assembly hg19 (GRCh37) to allow for the determination of the maximum count values for each detected transcript [31]. Data were sorted by maximum count numbers and compared between sample types to exclude concomitantly expressed genes and identify highly abundant and possibly specific body fluid markers. Four mRNA candidates were identified from this data set: haemoglobin delta (HBD) and solute carrier family 4, member 1 (SLC4A1) for circulatory blood, as well as matrix metallopeptidase 3 (MMP3) and stanniocalcin 1 (STC1) for menstrual fluid.

Two further candidate genes were selected from two gene expression databases (TIGER, PaGenBase) [32,33] based on their putative physiological function in the human body: transition protein 1 (TNP1) for spermatozoa and kallikrein-related peptidase 2 (KLK2) for seminal fluid which may be free of spermatozoa.

### Specificity Screening

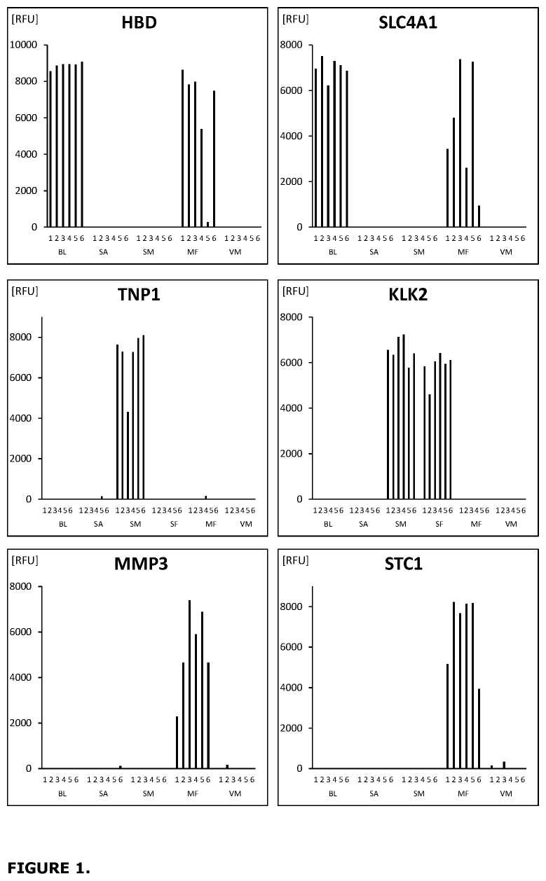

The expression profiles of the six body fluid marker candidates were evaluated by singleplex endpoint RT-PCR. Six samples per body fluid (50 μL circulatory blood and semen, whole buccal, menstrual and non-menstrual vaginal swabs) from various donors were amplified using 2 μL of cDNA synthesised from total RNA. When cross-reactive peaks were observed (TNP1, MMP3 and STC1, FIG. 1), the corresponding samples were rerun to verify signal reproducibility. Reverse transcription negative (RT-) controls omitting the RT enzyme were also run for each sample. All RT-controls were negative (data not shown).

### Haemoglobin Delta (HBD)

The haemoglobin delta or δ-globin (HBD) gene is part of the human β-globin gene cluster located on chromosome 11p15.5. Together with two alpha chains, two delta chains constitute the HbA₂ tetramer (α₂δ₂), which comprises about 2-3% of the total haemoglobin in adult humans [37]. The coding region of HBD has strong sequence homology with HBB, both of which are expressed in bone marrow and reticulocytes [28,29]. Mutations in the HBD gene can result in clinically insignificant δ-thalassaemia, characterised by a reduced ability of the body to produce HbA₂[37].

HBD mRNA was exclusively present in circulatory blood and menstrual fluid (FIG. 1). All circulatory blood and five of six menstrual fluid samples produced signals above 5000 RFU. The remaining menstrual sample (MF 5) produced a signal of 272 RFU, likely due to a lower blood content as this sample was taken on day 4 of the menstrual cycle and the donor reported only light bleeding. Accordingly, the obtained swab was lighter red in colour than the day 2 or 3 samples. All semen, buccal and vaginal material samples were negative (FIG. 1). These results demonstrate high abundance of HBD in blood and a specific expression pattern despite high sample input volumes.

Although HBD expression is known to reach only about 50% of that of HBB [37], our data show consistent and efficient detection of HBD mRNA and therefore demonstrate suitability of this marker for the identification of blood. The reduced expression is also advantageous given that the relatively strong and ubiquitous expression of HBB can lead to amplification from non-target body fluids [38,39]. While some of those observed signals may have been due to the presence of trace amounts of blood in a sample rather than true HBB expression, such findings clearly complicate the interpretation of results. Since HBD shows the same expression pattern as HBB, its reduced transcription rate is beneficial in this context as it increases marker specificity.

### Solute Carrier Family 4 (Anion Exchanger), Member 1 (Diego Blood Group) (SLC4A1)

SLC4A1, also known as anion exchanger 1 (AE1) or band 3, is located on chromosome 17q21-22, and is the main integral protein in the erythrocyte membrane, connecting the lipid bilayer to the protein network through interactions with ankyrin-1 and proteins 4.1 and 4.2 [40]. SLC4A1 also interacts with glycophorin A and haemoglobin [41]. The C-terminal domain functions as an anion exchanger, increasing the overall capacity of blood to transport CO₂ [40,41]. Numerous mutations in the SLC4A1 gene have been discovered, leading to conditions such as hereditary spherocytosis, southeast Asian ovalocytosis and hereditary acanthocytosis, all of which affect erythrocyte phenotype and result in minor to severe anaemia [40,41].

FIG. 1 shows that SLC4A1 mRNA was detected in all circulatory blood samples and two of six menstrual fluid samples at peak heights above 6000 RFU. The remaining menstrual fluid samples produced peaks of 3430 RFU (MF 1), 4804 RFU (MF 2), 2596 RFU (MF 4) and 937 RFU (MF 6), respectively. This may indicate slightly reduced expression of SLC4A1 in comparison to HBD, which on average produced 1.4-fold higher RFU from menstrual samples, however the difference was not statistically significant (Student's t-test, p>0.1). Furthermore, the primer concentration used for SLC4A1 (0.03 μM) was lower than that of HBD (0.05 μM) and different samples were used for the evaluation of both markers. Importantly, SLC4A1 was specific to samples containing blood and was not present in semen, buccal or vaginal material samples (FIG. 1).

### Transition Protein 1 (During Histone to Protamine Replacement) (TNP1)

Transition protein 1 (TNP1) has been mapped to chromosome 2q35-q36. Together with the larger TNP2, TNP1 replaces histones in the nuclei of elongating and condensing spermatids during spermiogenesis and is subsequently replaced by protamines [42]. TNP1 can destabilise nucleosomes and prevent DNA bending, and in turn promotes the repair of strand breaks by serving as an alignment factor [42]. Mutations in the promoter region of the TNP1 gene were found to reduce TNP1 expression and may contribute to male infertility [43].

Our results demonstrate strong expression of TNP1 in semen samples containing spermatozoa (FIG. 1). Notably, TNP1 was not detectable in six samples from an azoospermic donor or any of the circulatory blood and vaginal material samples. However, one saliva and one menstrual fluid sample produced peaks (147 and 152 RFU, respectively), although these were easily distinguished from semen samples, all of which exceeded 4300 RFU. The saliva and menstrual fluid samples were rerun to verify signal reproducibility and no peaks were observed, indicating that the initially observed signals likely resulted from amplification of trace amounts of TNP1 mRNA or non-specific primer binding. In both samples, replicate analysis clearly distinguished between cross-reactions and target signals.

### Kallikrein-Related Peptidase 2 (KLK2)

The gene encoding kallikrein-related peptidase 2 (KLK2), also referred to as human kallikrein 2, is located on chromosome 19q13.41. KLK2 is a serine protease synthesised by the prostate gland with high sequence identity to prostate-specific antigen (PSA/KLK3) [44]. It activates the zymogen forms of PSA and urokinase into their enzymatically active forms [44]. In addition, KLK2 possesses the ability to cleave semenogelins I and II, as well as fibronectin [45]. The enzymatic activity of KLK2 may be reversibly regulated by zinc ions, which are highest in the prostate and prostatic fluid [44].

As FIG. 1 shows, KLK2 mRNA was present in all semen samples tested, including six samples donated by an azoospermic individual. No cross-reactions with non-target body fluids were observed. All circulatory blood, buccal, menstrual fluid and vaginal material samples were negative (FIG. 1). Although previous studies have reported the presence of KLK2 mRNA in non-prostatic tissues, including salivary glands and endometrium [46], our findings demonstrate specificity of this mRNA to semen samples.

### Matrix Metallopeptidase 3 (MMP3)

Matrix metallopeptidases (MMPs) are a large family of zinc- or calcium-dependent endopeptidases which catabolise a wide range of substrates and thus regulate protein activity [47,48]. They engage in various roles during tissue degradation and remodelling processes, including menstruation [47,48]. Three members of this family, namely MMPs 7, 10 and 11, have been widely used as forensic menstrual fluid markers [36,38,48-51].

MMP3, also known as stromelysin-1 (mapped to 11q22.3) is another member of the MMP superfamily which is highly expressed during menstruation (FIG. 1). This enzyme is one of the key regulators of wound healing and scar formation [47]. Studies in mice have shown that defective MMP3 expression can lead to increased wound size, slowed wound healing and impaired scar contraction [47].

Our results identify MMP3 as a suitable menstrual fluid marker. This mRNA was strongly expressed on days 2 and 3 of the menstrual cycle. All six menstrual fluid samples produced peaks greater than 2000 RFU (FIG. 1). In addition, MMP3 mRNA was not detectable in circulatory blood and semen samples (FIG. 1). However, one buccal (113 RFU) and one vaginal material sample (day 19, 159 RFU) also produced peaks. When these samples were rerun, no signals were observed (data not shown).

In previous research, MMPs 7, 10 and 11 were introduced as markers specific for the detection of menstruum. Since then, multiple studies reported their expression during uterine phases outside of menstruation [48,51,52]. MMPs have also been detected in circulatory blood [39,51,52], saliva, semen and skin [52]. One study even suggested MMPI as a general vaginal secretion marker [53]. Here we also observed cross-reactions of MMP3 with saliva and vaginal material (FIG. 1). However, these signals were not reproducible and we conclude that they resulted from large sample input (i.e. whole swabs), leading to the amplification of trace amounts of MMP3 mRNA. Despite this, cross-reactive peaks were below 200 RFU and therefore clearly distinguishable from menstrual samples. Overall, the specificity of MMP3 to menstrual discharge is equal to or greater than that of MMPs 7, 10 or 11.

### Stanniocalcin 1 (STC1)

Stanniocalcin 1 (STC1) was originally described as a homodimeric glycoprotein in the corpuscles of bony fishes, where it regulates calcium and phosphate homeostasis [54].

In humans, the STC1 gene is located on chromosome 8p21.2, and the protein may also regulate intracellular calcium and/or phosphate levels as an autocrine or paracrine factor and thus contribute to bone formation [54,55]. In contrast to its function in fish, STC1 activity in humans is thought to be local rather than systemic due to its absence from the circulation [55]. Nevertheless, STC1 appears to be a pleiotropic factor, and other proposed functions include involvement in ischemia, angiogenesis, muscle contractility, as well as immune and inflammatory responses [54,55]. These processes are all known to take place in the endometrium before, during and after menstruation.

Our data confirm that STC1 mRNA is undetectable in circulatory blood samples (FIG. 1). In addition, no signals were obtained from buccal and semen samples, which is in agreement with earlier findings that STC1 mRNA is absent from seminal vesicles [55]. In this study STC1 was strongly expressed in menstrual samples (FIG. 1, average peak height 7703 RFU). However, two of six vaginal material samples also produced peaks (150 and 347 RFU, respectively). Both samples were rerun and no signals were observed (data not shown). Sample VM 1 was obtained on day 8 of the uterine cycle, which is the early post-menstrual phase. Therefore, this signal may be the result of residual trace amounts of STC1 mRNA which were collected during swabbing. Sample VM 3, in contrast, was taken on day 19 of the uterine cycle from a different individual. This donor used a hormonal contraceptive at the time of sample donation, which could have had an effect on STC1 expression. STC1 expression in ovaries has been reported [55] and it appears that cross-reactions are most likely obtained from vaginal samples. Further research could address whether the menstrual cycle stage during which a sample is obtained or the use of contraceptives influence STC1 expression.

### Comparison to Existing Markers

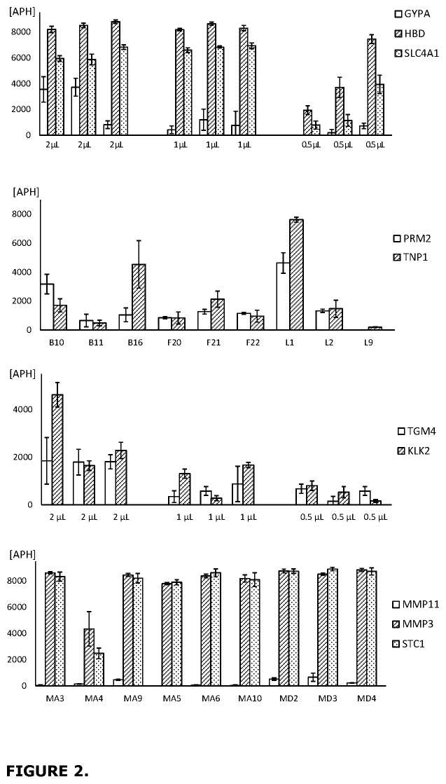

The sensitivity of the six novel body fluid candidates was compared to corresponding well-characterised markers published previously [36]. HBD and SLC4A1 were compared to Glycophorin A (GYPA), TNP1 to protamine 2 (PRM2), KLK2 to transglutaminase 4 (TGM4), and MMP3 and STC1 to MMP11. As FIG. 2 illustrates, all new mRNAs produced higher average peak heights (APH) from their respective target body fluids than corresponding markers. Both HBD and SLC4A1 were significantly more sensitive for the detection of blood at the tested primer concentration of 0.2 μM than GYPA (Student's t-test, p<0.0005 for HBD and p<0.005 for SLC4A1). The increased sensitivity of TNP1 to semen samples at a primer concentration of 0.05 μM was also statistically significant (p<0.05). The lowest p-values, however, were obtained for the comparison of MMP11 to MMP3 (p<5·10⁻²¹) and STC1 (p<5·10⁻¹⁷). These findings demonstrate an extremely significant enhancement in detection sensitivity compared to MMP11. Both MMP3 and STC1 mRNAs are therefore much more abundant in the menstruating endometrium than MMP11, while displaying the same expression pattern [36,38,51]. Only the increase in peak height for KLK2 did not reach statistical significance, although 67% of semen samples produced higher KLK2 signals compared to TGM4.

### CONCLUSION

This work evaluated the expression of six novel mRNAs for forensic body fluid identification by singleplex endpoint reverse transcription (RT-PCR). All candidates were highly abundant in their respective target body fluid type compared to other bodily sources. Haemoglobin delta (HBD) and solute carrier family 4, member 1 (SLC4A1) can be used to confirm the presence of circulatory blood. Transition protein 1 (TNP1) mRNA was present in semen which contains spermatozoa, while kallikrein-related peptidase 2 (KLK2) mRNA was exclusive to seminal fluid regardless of spermatozoa presence. Matrix metallopeptidase 3 (MMP3) and stanniocalcin 1 (STC1) can be used to identify menstrual fluid samples.

All six candidate mRNAs showed increased detection sensitivity compared to corresponding known markers [36]. With the exception of KLK2, the increased average peak height reached statistical significance up to an extreme p-value of 5·10⁻²¹ for MMP3 compared to MMP11. Both MMP3 and STC1 mRNA were significantly more abundant in the endometrium during menstruation than MMP11 and can therefore improve the successful identification of a blood stain resulting from menses. In particular the detection of STC1 can be useful for the discrimination between circulatory blood and menstrual fluid due to its absence from the circulatory system [55]. In this study, STC1 mRNA expression was only observed in menstrual and vaginal material samples, even when the primer concentration was raised to 0.4 μM (data not shown). A time-wise study could help determine whether STC1 expression varies between stages of the uterine cycle or between women who use hormonal contraceptives and those who do not.

Single cross-reactions were observed for TNP1 with saliva and menstrual fluid, for MMP3 with saliva and vaginal material, and for STC1 with two non-menstrual vaginal samples (FIG. 1). These peaks remained below 350 RFU in all cases and were therefore easily distinguishable from target body fluid signals. In addition, cross-reactions were not reproducible, hence our data support earlier findings that technical replicates may be useful for mRNA result interpretation [56]. Moreover, it should be kept in mind that the volume of extracted body fluid or RNA/cDNA input amount, respectively, plays a major role in the occurrence of cross-reactive peaks. This study used large body fluid volumes (50 μL or a whole swab) and undiluted cDNA samples in order to uncover trace expression and explore the limits of marker specificity. In view of this, cross-reactions were expected, however all non-target signals we observed were of lower peak height than target signals and were non-reproducible. Additionally, in forensic casework, samples are typically of small size, degraded or otherwise compromised [31,34], thus limiting the amount of RNA and cDNA that can be obtained from the samples. Therefore, at the primer concentrations used here (Table 1), we are confident that cross-reactions with non-target body fluids are kept at a minimum, in particular when combined with controlled RNA or cDNA input amounts, stringent PCR conditions and suitable interpretation guidelines [39,52,57,58].

The simultaneous assessment of multiple mRNAs per body fluid can help avoid false positives, since it is less likely that all typed markers would falsely indicate the presence of a certain body fluid [59]. Hence, the novel mRNAs characterised here can greatly increase the probative value of mRNA results by expanding the panel of useful forensic body fluid markers.

### REFERENCES

[1] Untergasser A, Cutcutache I, Koressaar T, Ye J, Faircloth B C, Remm M, et al. Primer3—new capabilities and interfaces. Nucleic Acids Research. 2012;40:e115-e.

[2] Ye J, Coulouris G, Zaretskaya I, Cutcutache I, Rozen S, Madden TL. Primer-BLAST: a tool to design target-specific primers for polymerase chain reaction. BMC Bioinformatics. 2012;13:134.

[3] Dieffenbach C, Lowe T, Dveksler G. General concepts for PCR primer design. PCR Methods and Applications. 1993;3:S30-S7.

[4] Hyndman D L, Mitsuhashi M. PCR primer design. PCR Protocols: Springer; 2003. p. 81-8.

[5] Mann T, Humbert R, Dorschner M, Stamatoyannopoulos J, Noble W S. A thermodynamic approach to PCR primer design. Nucleic Acids Research. 2009;37:e95-e.

[6] Peters I R, Helps C R, Hall E J, Day M J. Real-time RT-PCR: considerations for efficient and sensitive assay design. Journal of Immunological Methods. 2004;286:203-17.

[7] Rozen S, Skaletsky H. Primer3 on the WWW for general users and for biologist programmers. Bioinformatics Methods and Protocols: Springer; 1999. p. 365-86.

[8] Ginzinger DG. Gene quantification using real-time quantitative PCR: an emerging technology hits the mainstream. Experimental Hematology. 2002;30:503-12.

[9] Kovárová M, Dráker P. New specificity and yield enhancer of polymerase chain reactions. Nucleic Acids Research. 2000;28:e70-e.

[10] Lebedev A V, Paul N, Yee J, Timoshchuk V A, Shum J, Miyagi K, et al. Hot start PCR with heat-activatable primers: a novel approach for improved PCR performance. Nucleic Acids Research. 2008;36:e131-e.

[11] Mikeska T, Dobrovic A. Validation of a primer optimisation matrix to improve the performance of reverse transcription-quantitative real-time PCR assays. BMC Research Notes. 2009;2:112.

[12] Reynisson E, Josefsen M H, Krause M, Hoorfar J. Evaluation of probe chemistries and platforms to improve the detection limit of real-time PCR. Journal of Microbiological Methods. 2006;66:206-16.

[13] Huggett J, Bustin S A. Standardisation and reporting for nucleic acid quantification. Accreditation and Quality Assurance. 2011;16:399-405.

[14] Huggett J, Dheda K, Bustin S, Zumla A. Real-time RT-PCR normalisation; strategies and considerations. Genes & Immunity. 2005;6:279-84.

[15] Ashlock D, Wittrock A, Wen T-J. Training finite state machines to improve PCR primer design. Computational Intelligence, Proceedings of the World on Congress on: IEEE; 2002. p. 13-8.

[16] Latorra D, Arar K, Hurley J M. Design considerations and effects of LNA in PCR primers. Molecular and Cellular Probes. 2003;17:253-9.

[17] Tichopad A, Dzidic A, Pfaffl M W. Improving quantitative real-time RT-PCR reproducibility by boosting primer-linked amplification efficiency. Biotechnology Letters. 2002;24:2053-6.

[18] Afonina I, Ankoudinova I, Mills A, Lokhov S, Huynh P, Mahoney W. Primers with 5′flaps improve real-time PCR. BioTechniques. 2007;43:770.

[19] Sachs AB. Messenger RNA degradation in eukaryotes. Cell. 1993;74:413-21.

[20] Houseley J, Tollervey D. The many pathways of RNA degradation. Cell. 2009;136:763-76.

[21] Frazao C, McVey C E, Amblar M, Barbas A, Vonrhein C, Arraiano C M, et al. Unravelling the dynamics of RNA degradation by ribonuclease II and its RNA-bound complex. Nature. 2006;443:110-4.

[22] van Hoof A, Parker R. Messenger RNA degradation: beginning at the end. Current Biology. 2002;12:R285-R7.

[23] Christodoulou D C, Gorham J M, Herman D S, Seidman J. Construction of normalized RNA-seq libraries for Next-Generation Sequencing using the crab duplex-specific nuclease. Current Protocols in Molecular Biology. 2011:4.12. 1-4.1.

[24] Fleige S, Waif V, Huch S, Prgomet C, Sehm J, Pfaffl M W. Comparison of relative mRNA quantification models and the impact of RNA integrity in quantitative real-time RT-PCR. Biotechnology Letters. 2006;28:1601-13.

[25] Rowley J W, Oler A J, Tolley N D, Hunter B N, Low E N, Nix D A, et al. Genome-wide RNA-seq analysis of human and mouse platelet transcriptomes. Blood. 2011;118:e101-e11.

[26] Schroeder A, Mueller O, Stocker S, Salowsky R, Leiber M, Gassmann M, et al. The RIN: an RNA integrity number for assigning integrity values to RNA measurements. BMC Molecular Biology. 2006;7:3.

[27] Auer H, Lyianarachchi S, Newsom D, Klisovic MI. Chipping away at the chip bias: RNA degradation in microarray analysis. Nature Genetics. 2003;35:292-3.

[28] Fleige S, Pfaffl M W. RNA integrity and the effect on the real-time qRT-PCR performance. Molecular Aspects of Medicine. 2006;27:126-39.

[29] Romero I G, Pai A A, Tung J, Gilad Y. RNA-seq: Impact of RNA degradation on transcript quantification. BMC Biology. 2014;12:42.

[30] Antonov J, Goldstein D R, Oberli A, Baltzer A, Pirotta M, Fleischmann A, et al. Reliable gene expression measurements from degraded RNA by quantitative real-time PCR depend on short amplicons and a proper normalization. Laboratory Investigation. 2005;85:1040-50.

[31] Lin M-H, Jones D F, Fleming R. Transcriptomic analysis of degraded forensic body fluids. Forensic Science International: Genetics. 2015;17:35-42.

[32] Liu X, Yu X, Zack D J, Zhu H, Qian J. TIGER: a database for tissue-specific gene expression and regulation. BMC Bioinformatics. 2008;9:271.

[33] Pan J-B, Hu S-C, Shi D, Cai M-C, Li Y-B, Zou Q, Ji Z-L. PaGenBase: a pattern gene database for the global and dynamic understanding of gene function. PLOS ONE. 2013;8(12):e80747.

[34] Lin M-H, Albani P P, Fleming R. Degraded RNA transcript stable regions (StaRs) as targets for enhanced forensic RNA body fluid identification. Forensic Science International: Genetics. 2016;20:61-70.

[35] Ye J, Coulouris G, Zaretskaya I, Cutcutache I, Rozen S, Madden T L. Primer-BLAST: a tool to design target-specific primers for polymerase chain reaction. BMC Bioinformatics. 2012;13:134.

[36] Fleming R I, Harbison S. The development of a mRNA multiplex RT-PCR assay for the definitive identification of body fluids. Forensic Science International: Genetics. 2010;4:244-56.

[37] Steinberg M H, Rodgers G P. HbA2: biology, clinical relevance and a possible target for ameliorating sickle cell disease. British Journal of Haematology. 2015; doi: 10.1111/bjh.13570. [Epub ahead of print].

[38] Lindenbergh A, de Pagter M, Ramdayal G, Visser M, Zubakov D, Mayser M, Sijen T. A multiplex (m)RNA-profiling system for the forensic identification of body fluids and contact traces. Forensic Science International: Genetics. 2012;6:565-577.

[39] Roeder A D, Haas C. mRNA profiling using a minimum of five mRNA markers per body fluid and a novel scoring method for body fluid identification. International Journal of Legal Medicine. 2013;127:707-721.

[40] Iolacson A, Perrotta S, Stweart G W. Red blood cell membrane defects. Reviews in Clinical & Experimental Hematogoly. 2003;7:22-56.

[41] Williamson R C, Toye A M. Glycophorin A: band 3 aid. Blood Cells, Molecules, and Diseases. 2008;41:35-43.

[42] Meistrich M L, Mohaparta B, Shirley C R, Zhao M. Roles of transition nuclear proteins. Chromosoma. 2003;111:483-488.

[43] Miyagawa Y, Nishimura H, Tsujimura A, Matsuoka Y, Matsumiya K, Okuyama A, Nishimune Y, Tanaka H. Single-nucelotide polymorphisms and mutation analyses of the TNP1 and TNP2 genes of fertile and infertile human male populations. Journal of Andrology. 2005;26:779-786.

[44] Lövgren J, Airas K, Lilja H. Enzymatic action of human glandular kallikrein 2 (hK2). European Journal of Biochemistry. 1999;262:781-789.

[45] Clements J A, Willemsen M N, Myers S A, Dong Y. The tissue kallikrein family of serine proteases: functional roles in human disease and potential as clinical biomarkers. Critical Reviews in clinical Laboratory Sciences. 2004;41(3):265-312.

[46] Lövgren J, Valtonen-André C, Mersal K, Lilja H, Lundwall Å. Measurement of prostate-specific antigen and human glandular kallikrein in different body fluids. Journal of Andrology. 1999;20(3):348-355.

[47] Gill S, Parks W C. Metalloproteinases and their inhibitors: regulators of wound healing. The International Journal of Biochemistry & Cell Biology. 2008;40:1334-1347.

[48] Bauer M, Patzelt D. Identification of menstrual blood by real time RT-PCR: technical improvements and the practical value of negative test results. Forensic Science International. 2008;174:54-58.

[49] Juusola J, Ballantyne J. Multiplex mRNA profiling for the identification of body fluids. Forensic Science International. 2005;152:1-12.

[50] Juusola J, Ballantyne J. mRNA profiling for body fluid identification by multiplex quantitative RT-PCR. Journal of Forensic Sciences. 2007;52(6):1252-1262.

[51] Haas C, Klesser B, Maake C, Bär W, Kratzer A. mRNA profiling for body fluid identification by reverse transcription endpoint PCR and realtime PCR. Forensic Science International: Genetics. 2009;3:80-8.

[52] Van den Berge M, Carracedo A, Gomes I, Graham E A M, Haas C, Hjort B, et al. A collaborative European exercise on mRNA-based body fluid/skin typing and interpretation of DNA and RNA results. Forensic Science International: Genetics. 2014;10:40-8.

[53] Park S-M, Park S-Y, Kim J-H, Kang T-W, Park J-L, Woo K-M, et al. Genome-wide mRNA profiling and multiplex quantitative RT-PCR for forensic body fluid identification. Forensic Science International: Genetics. 2013;7:143-150.

[54] Yoshiko Y, Aubin J E. Stanniocalcin 1 as a pleiotropic factor in mammals. Peptides. 2004;25:1663-1669.

[55] Yeung B H Y, Law A Y S, Wong C K C. Evolution and roles of stanniocalcin. Molecular and Cellular Endocrinology. 2012;349:272-280.

[56] Van den Berge M, Bhoelai B, Harteveld J, Matai A, Sijen T. Advancing forensic RNA typing: On non-target secretions, a nasal mucosa marker, a differential co-extraction protocoal and the sensitivity of DNA and RNA profiling. Forensic Science International: Genetics. 2016;20:119-129.

[57] Haas C, Hanson E, Bär W, Banemann R, Bento AM, Berti A, et al. mRNA profiling for the identification of blood-results of a collaborative EDNAP exercise. Forensic Science International: Genetics. 2011;5:21-26.

[58] Haas C, Hanson E, Anjos M J, Banemann R, Berti A, Borges E, et al. RNA/DNA co-analysis from human saliva and semen stains—results of a third collaborative EDNAP exercise. Forensic Science International: Genetics. 2013;7:230-239.

[58] Haas C, Hanson E, Kratzer A, Bär W, Ballantyne J. Selection of highly specific and sensitive mRNA biomarkers for the identification of blood. Forensic Science International: Genetics. 2011;5:449-458.

### SEQUENCE LISTING

(polynucleotide, Beta-hemoglobin (HBB))
SEQ ID NO: 1
CACACTGAGT GAGCTGCACT GTGACAAGCT GCACGTGGAT CCTGAGAACT TCAGGCTCCT GGGCAACGTG
CTGGTCTGTG TGCTGGCCCA TCACTTTGGC AAAGA
(polynucleotide, glycophorin A (GYPA))
SEQ ID NO: 2
GAACCAGAGA TAACACTCAT TATTTTTGGG GTGATGGCTG GTGTTATTGG AACGATCCTC TTAATTTCTT
ACGGTATTCG CCGACTGATA AAGAAAAGCC CATCTGATGT AAAACCTCTC CCCTCACCTG ACACAGACGT
GCCTTTAAGT TCTGTTGAAA TAGAAAATCC AGA
(polynucleotide, delta-aminolevulinate synthase (ALAS2))
SEQ ID NO: 3
GACATCAT CTCTGGAACT CTTGGCAAGG CCTTTGGCTG TGTGGGCGGC TACATTGCCA GCACCCGTGA
CTTGGTGGAC ATGGTGCGCT CCTATGCTGC AGGCTTCATC TTTACCACTT CTCTGCCCCC CATGGTGCTC
TCTGGAGCTC TAGAATCTGT GCGGCTGCTC AAGGGAGAGG AGGGCCAAGC CCTGAGGCGA
GCCCACCAGC GCAATGTCAA GCACATGCGC CAG CTACTCA TGGACAGGGG CCTTCCTGTC ATCCCCTGCC
CCAGCCACAT CATCCCCATC CGGGTGGGCA ATGCAGCACT CAACAGCAAG CTCTGTGATC TCCTGCTCTC
CAAGCATGGC ATCTATGTGC AGGCCATCAA CTACCCAACT GTCCCCCGGG GTGAAGAGCT CCTGCGCTTG
GCACCCTCCC CCCACCACAG CCCT
(polynucleotide, solute carrier family 4 (anion exchanger), member 1
(Diego blood group) (SLC4A1))
SEQ ID NO: 4
ATGATGGAGGA GAATCTGGAG CAGGAGGAAT ATGAAGACCC AGACATCCCC GAGTCCCAGA
TGGAGGAGCC GGCAGCTCAC GACACCGAGG CAACAGCCAC AGACTACCAC ACCACATCAC
(polynucleotide, pro-platelet basic protein (chemokine (C-X-C motif) ligand
7) (PPBP)
SEQ ID NO: 5
GAGG CTCGTGAGCA GGGACCCGCG GTGCGGGTTA TGCTGGGGGC TCAGATCACC GTAGACAACT
GGACACTCAG GACCACG CCA TGGAGGAGCT GCAGGATGAT TATGAAGACA TGATGGAGGA
GAATCTGGAG CAGGAGGAAT ATGAAGACCC AGACATCCCC GAGTCCCAGA TGGAGGAGCC
GGCAGCTCAC GACACCGAGG CAACAGCCAC AGACTACCAC ACCACATCAC ACCCGGGTA
(polynucleotide, hemoglobin delta (HBD))
SEQ ID NO: 6
CACCATGGT GCATCTGACT CCTGAG GAGA AGACTGCTGT CAATGCCCTG TGGGGCAAAG TGAACGTGGA
TGCAGTTGGT GGTGAGGCCC TGGGCAGATT ACTG
(polynucleotide, hemoglobin delta (HBD))
SEQ ID NO: 7
GCACTGTGAC AAGCTGCACG TGGATCCTGA GAACTTCAGG CTCTTGGGCA ATGTGCTGGT GTGTGTGCTG
GCCCG
(polynucleotide, hemoglobin subunit alpha (HBA)
SEQ ID NO: 8
GCCC AACGCGCTGT CCGCCCTGAG CGACCTGCAC GCGCACAAGC TTCGGGTGGA CCCGGTCAAC
TTCAAGCTCC TAAGCCACTG CCTGCTGGTG ACCCTGGCCG CCCACCTCCC CGCCGAGTTC
(polynucleotide, matrix metallopeptidase 10 (MMP10))
SEQ ID NO: 9
GAAAGG ACAGTAATCT CATTGTTAAA AAAATCCAAG GAATGCAGAA GTTCCTTGGG TTGGAGGTGA
CAGGGAAGCT AGACACTGAC ACTCTGGAGG TGATGCGCAA GCCCAGGTGT GGAGTTCCTG ACGTTGGTCA
CTTCAGCTCC TTTCCTGGCA TGCCGAAGTG GAGGAAAACC CACCTTACAT ACAGGATTGT GAATTATACA
CCAGATTTGC CAAGAGATGC TGTTGATTCT GCCATTGAGA AAGCTCTGAA AGTCTGGGAA GAGGTGACTC
CACTCACATT CTCCAGGCTG TATGAAGGAG AGGCTGATAT AATGATCTCT TTTGCAGTTA AAGAACATGG
AGACTTTTAC TCTTTTGATG GCCCAGGACA CAGTTTGGCT CATGCCTACC CACCTGGACC TGGGCTTTAT
GGAGATATTC ACTTTGATGA TGATGAAAAA TGGACAGAAG ATGCATCAGG CACCAATTTA TTCCTCGTTG
CTGCTCATGA ACTTGGCCAC TCCCTGGGGC TCTTTCACTC AGCCAACACT GAAGCTTTGA TGTACCCACT
CTACAACTCA TTCACAGAGC TCGCCCAGTT CCGCCTTTCG CAAGATGATG TGAATGGCAT TCAGTCTCTC
TACG
(polynucleotide, matrix metallopeptidase 11 (MMP11))
SEQ ID NO: 10
ACAGACCTGC TGCAGGTGGC AGCCCATGAA TTTGGCCACG TGCTGGGGCT GCAGCACACA ACAGCAGCCA
AGGCCCTGAT GTCCGCCTTC TACACCTTTC GCTACCCACT GAGTCTCAGC CCAGATGACT GCAGGGGCGT
TCAACACCTA TATGGCCAGC CCTGGCCCAC TGTCACCTCC AGGACCCCAG CCCTGG
(polynucleotide, matrix metallopeptidase 3 (MMP3))
SEQ ID NO: 11
GATTG TGAATTATAC ACCAGATTTG CCAAAAGATG CTGTTGATTC TGCTGTTGAG AAAGCTCTGA
AAGTCTGGGA AGAGGTGACT CCACTCACAT TCTCCAGGCT GTATGAAGGA GAGGCTGATA TAATGATCTC
TTTTGCAGTT AGAGAACATG GAGACTTTTA CCCTTTTGAT GGACCTGGAA ATGTTTTGGC CCATGCCTAT
GCCCCTGGGC CAGGGATTAA TGGAGATGCC CACTTTGATG ATGATGAACA ATGGACAAAG GATACAACAG
GGACCAATTT ATTTCTCGTT GCTGCTCATG AAATT
(polynucleotide, plasminogenin activator urokinase receptor (PLAUR))
SEQ ID NO: 12
TCCTGGA GCTTGAAAAT CTGCCGCAGA ATGGCCGCCA GTGTTACAGC TGCAAGGGGA ACAGCACCCA
TGGATGCTCC TCTGAAGAGA CTTTCCTCAT TGACTGCCGA GGCCCCATGA ATCAATGTCT GGTAGCCACC
GGCACTCACG AACCGAAAAA CCAAAGCTAT ATGGTAAGAG GCTGTGCAAC CGCCTCAATG TGCCAACATG
CCCACCTGGG TGACGCCTTC AGCATGAACC ACATTGATGT CTCCTGCTGT ACTAAAAGTG GCTGTAACCA
CCCAGACCTG GATGTCCAGT ACCGCAGTGG GGCTGCTCCT CAGCCTGGCC CTGCCCATCT CAGCCTCACC
ATCACCCTGC TAATGACTGC CAGACTGTGG GGAGGCACTC TCCTCTGGAC CTAAAC
(polynucleotide, stanniocalcin 1 (STC1))
SEQ ID NO: 13
A GACACAGTCA GCACAATCAG AGACAGCCTG ATGGAGAAAA TTGGGCCTAA CATGGCCAGC
CTCTTCCACA TCCTGCAGAC AGACCACTGT GCCCAAACAC ACCCACGAGC TGACTTCAAC AGGAGACGCA
CCAATGAGCC GCAGAAGCTG AAAGTCCTCC TCAGGAACCT CCGAGGTGAG GAGGACTCTC CCTCCCACAT
CAAACGCACA TCCCATGAGA GTGCATAACC AGGGAGAGGT TATTCACAAC CTCACCAAAC TAGTATCATT
TTAGGGGTGT TGACACACCA GTTTTGAGTG TACTGTGCCT GGTTTGATTT TTTTAAAGTA GTTCCTATTT
TCTATCCCCC TTAAAGAAAA TTGCATGAAA CTAGGCTTCT GTAATCAATA TCCCAACATT CTGCAATGGC
AGCATTCCCA CCAACAAAAT CCATGTGACC ATTCTGCCTC TCCTCAGGAG AAAGTACCCT CTTTTACCAA
CTTCCTCTGC CATGT
(polynucleotide, transglutaminase 4 (TGM4))
SEQ ID NO: 14
T TGCCTAACAC AGGCAGAATT GGCCAGCTAC TTGTCTGCAA TTGTATCTTC AAGAATACCC TGGCCATCCC
TTTGACTGAC GTCAAGTTCT CTTTGGAAAG CCTGGGCATC TCCTCACTAC AGACCTCTGA CCATGGGACG
GTGCAGCCTG GTGAGACCAT CCAATCCCAA ATAAAATGCA CCCCAATAAA AACTGGACCC AAGAAATTTA
TCGTCAAGTT AAGTTCCAAA CAAGTGAAAG AGATTAATGC TCAGAAGATT GTTCTCATCA CCAAGTAGCC
TTGTCTGATG CTGTGGAGCC TTAGTTGAGA TTTCAGCATT TCCTACCTTG TGCTTAGCTT TCAGATTATG
GATGATTAAA TTTGATGA
(polynucleotide, semenogelin 2 (SEMG2))
SEQ ID NO: 15
ATGAAGTCC ATCATCCTCT TTGTCCTTTC CCTGCTCCTT ATCTTGGAGA AGCAAGCAGC TGTGATGGGA
CAAAAAGGTG GATCAAAAGG CCAATTGCCA AGCGGATCTT CCCAATTTCC ACATGGACAA AAGGG
(polynucleotide, semenogelin 1 (SEMG1))
SEQ ID NO: 16
AAAT CCAGGCACCA AATCCTAAGC AAGAGCCATG GCATGGTGAAAATGCAAAAG GAGAGTCTGG
CCAATCTACA AATAGAGAAC AAGACCTACT CAGTCATGAACAAAAAGGCA GACACCAACA TGGATCTCAT
GGGGGATTGG ATATTGTAAT TATAGAGCAGGAAGATGACA GTGATCGTCATTTGGCACAA CATCTTAACA
ACGACCGAAA CCCATTA
(polynucleotide, microseminoprotein beta (MSMB))
SEQ ID NO: 17
GTACCTGTCT ATAAGGAGTC CTGCTTATCA CAATGAATGT TCTCCTGGGC AGCGTTGTGATCTTTGCCAC
CTTCGTGACT TTATGCAATG CATCATGCTA TTTCATACCT AATGAGGGAGTTCCAGGAGA TTCAACCAG
(polynucleotide, spermatogenesis associated 42 (SPATA42))
SEQ ID NO: 18
ACTGGGAATC TGATGGACTC AATTAAGAAT TTCTACAGAT GGGAAAACCA AAACTCCTTA GTGGCAAGAG
GCCAAAGATG GTCAGCGAAT TGTTGTTTCC G
(polynucleotide, protamine 1 (TNP1))
SEQ ID NO: 19
TGCTC ACAGGTTGGC TGGCTCAGCC AAGGTGGTGCCCTGCTCTGA GCATTCAGGC CAAGCCCATC
CTGCACCATG GCCAGGTACA GATGCTGTCGCAGCCAGAGC CGGAGCAGAT ATTACCGCCA GAGACAAAGA
AGTCGCAGAC GAAGGAGGCGGAGCTGCCAG ACACGGAGGA GAGC
(polynucleotide, histatin 3 (HTN3))
SEQ ID NO: 20
TTCACATCGAGGCTATAGAT CAAATTATCT GTATGACAAT TGATATCTTC AGTAATCACG GGGCATGATT
ATGGAGGTTT
(polynucleotide, statherin (STATH))
SEQ ID NO: 21
GTA TGGCCCTTAT CAGCCAGTTCCAGAACAACC ACTATACCCA CAACCATACC AACCACAATA
CCAACAATAT ACCTTTTAAT ATCATCAGTA ACTGCAGGAC ATGATTATTG AGGCTTGATT GGCAAATACG
ACTTCTACAT CCATATTCTC ATCTTTCATA CCATATCACA CTACTACCAC TTTT
(polynucleotide, follicular dendritic cell secreted protein (FDCSP))
SEQ ID NO: 22
ATCAGTGA CAGCGATGAA TTAGCTTCAG GGTTTTTTGT GTTCCCTTAC CCATATCCAT TTCGCCCACT
TCCACCAATT CCATTTCCAA GATTTCCATG GTTTAGACGT AATTTTCCTA TTCCAATACC TGAATCTGCC
CCTACAACTC CCCTTCCTAG CGAAAAGTAA ACAAGAAGGA AAAGTCACGA TAA
(polynucleotide, proline-rich protein BstNI subfamily 4 (PRB4))
SEQ ID NO: 23
AAACCAGTCCCAAGGTCCC CCACCTCCTC CAG GAAAGCC AGAAGGACGA CCCCCACAAG
GAGGCAACCAGTCCCAAGGT CCCCCACCTC ATCCAGGAAA GCCAGAAAGA CCACCCCCAC
AAGGAGGAAACCA
(polynucleotide, proline-rich protein BstNI subfamily 4 (PRB4))
SEQ ID NO: 24
TGGA AAGCCACAAG GCCCACCCCCAGCAGGAGGC AATCCCCAGC AGCCTCAGGC ACCTCCTGCT
GGAAAGCCCC AGGGGCCACCTCCACCTCCT CAAGGGGGCAGGCCACCCAG ACCTGCCCAG GGACAACAGC
CTCCCCAGTAATCTAGGATT CAATGACAG
(polynucleotide, metallothionein 1 (MT1X))
SEQ ID NO: 25
T TGGCTCCTGT GCCTGTGCCGGCTCCTGCAA ATGCAAAGAG TGCAAATGCA CCTCCTGCAA
(polynucleotide, metallothionein 1 (MT1X))
SEQ ID NO: 26
GCTCCTGCT GCCCTGTGGG CTGTGCCAAG TGTGCCCAGG GCTGCATCTG CAAAGGGACG
TCAGACAAGTGCAGCTGCTG TGCCTGATGC CAGGACAGCT GTGCTCTCAG ATGTAAATAG
AGCAACCTATATAA
(polynucleotide, uridine phosphorylase 1 (UPP1))
SEQ ID NO: 27
GCTTGGTGAG GTGACTCGCG GTCGCGGGTGACTCGCCGGC AGGACACTGC CTGGAACGCC TGGAGCGCCT
CCCACTGCAG ACGTCTGTCCGCCTCCAGCC GCTCTCCTCT GACGGGTCCT GCCTCAGTTG GCGGAATGGC
GGCCACGGGAGCCAATGCAG AGAAAGCTGA AAGTCAC
(polynucleotide, uridine phosphorylase 1 (UPP1))
SEQ ID NO: 28
TTTCAATC TCACCACTAG CAGACACAATTTCCCAGCCT TGTTTGGAGA TGTGAAGTTTGTGTGTGTTG
GTGGAAGCCC CTCCCGGATGAAAGCCTTCA TCAGGTGCGT TGGTGCAGAG
(polynucleotide, uridine phosphorylase 1 (UPP1))
SEQ ID NO: 29
GC AGATTGTCCT GGGGAAGCGG GTCATCCGGA AAACGGACCT TAACAAGAAGCTGGTGCAGG
AGCTGTTGCT GTGTTCTGCA GAGCTGAGCG AGTTCACCAC AGTGGTGGGG AACACCATGT GCACCTTGGA
CTTCTATGAA GGGCAAGGCC GTCTGGATGG GGCTCTCTGCTCCTACACGG AGAAGGACAA GCAGGCGTAT
CTGGAGGCAG CCTATGCAGC CGGCGTCCGCAATATCGAGA TGGAGTCCTC GGTGTTTGCC GCCATGTGCA
GCGCCTGCGG CCTCCAAGCGGCCGTGGTGT GTGTCACCCT CCTGAACCGC CTGGAAGGGG ACCAGATCAG
CAGCCCTCGCA
(polynucleotide, chemokine (C-X-C motif) ligand 8 (CXCL8))
SEQ ID NO: 30
CCTGATTTC TGCAGCTCTG TGTGAAGGTG CAGTTTTGCC AAGGAGTGCTAAAGAACTTA GATGTCAGTG
(polynucleotide, chemokine (C-X-C motif) ligand 8 (CXCL8))
SEQ ID NO: 31
GCGCCA ACACAGAAAT TATTGTAAAGCTTTCTGATG GAAGAGAGCT CTG
(polynucleotide, myozenin 1 (MYOZ1))
SEQ ID NO: 32
AGTGGGAGA ATCCCAAAGG CCTTTTCCCTCCTTCCTGAG CCTCCGGGCA AGGAGGGAGG GATCTTGGTT
CCAGGGTCTC AGTACCCCCTGTGCCATTTG AGCTGCTTGC GCTCATCATC TCTATTAATA ACCAACTTCC
CTCCCCCACTGCCAGTGCTG CCCCCACGCC TGCCCAGCTC GTGTTCTCCG GTC
(polynucleotide, defensin beta 4A (DEFB4A))
SEQ ID NO: 33
CAGGAC CTTTATAAGG TGGAAGGCTT GATGTCCTCC CCAGACTCAGCTCCTGGTGA AGCTCCCAGC
CATCAGCCAT GAGGGTCTTG TATCTCCTCT TCTCGTTCCTCTTCATATTC CTGATGCCTC TTCCAGGTGT
(polynucleotide, tyrosine kinase binding protein (TYROBP))
SEQ ID NO: 34
GGGGGGA CTTGAACCCT GCAGCAGGCT CCTGCTCCTGCCTCTCCTGC TGGCTGTAAG TGGTCTCCGT
CCTGTCCAGG CCCAGGCCCA GAGCGATTGCAGTTGCTCTA CGGTGAGCCC GGGCGTGCTG GCAGGGATCG
TGATGGGAGA CCTGGTGCTGACAGTGCTC
(polynucleotide, tyrosine kinase binding protein (TYROBP))
SEQ ID NO: 35
GCCCGAA TCATGACAGT CAGCAACATG ATACCTGGAT CCAGCCATTC CTGAAGCCCA CCCTGCACCT
CATTCCAACT CCTACCGCGA TACAGACCCA CAGAGTGCCA TCCCTGAGAG ACCAGA
(polynucleotide, cytochrome P450 family 2 subfamily B member 7,
pseudogene (CYP2B7P1))
SEQ ID NO: 36
CACT GCTCTCCGTGACCCACACTA CTTTGAAAAA CCAGACGCCT TCAATCCTGA CCACTTTCTG
GATGCCAATGGGGCACTGAA AAAGAATGAA GCTTTTATCC CCTTCTCCTT AGGGAAGCGG
ATTTGTCTTGGTGAAGGCAT TGCCCGTGCG GAATTGTTCC TCTTCTTCAC CACCATCCTC CAGAACTTCT
CCGTGGCCAG CCCCGTGGCT CCTGAAGACA TCGATCTGAC ACCCCAGGAG TGTGGTGTGGGCAAAATACC
CCCAACATAC CAGATCTGCT TCCTGCCCCG CTGAAGGGGC TGAGGGAAGGGGGTCAAAGG ATTC
(polynucleotide, U6 small nuclear 1149 (RNU6-1149P))
SEQ ID NO: 37
GCTCACTT TGGCAGCACA TATAACTAAA ATTGGAATGC TGCAGAGAAG ATTAGCATGG CCCCTACATT
TAAA
(polynucleotide, small proline rich protein 2G (SPRR2G))
SEQ ID NO: 38
TGGCT CTTCTTACTC CCAGGACTCC ATCATCTTCCCTTCAGCTGT AGTGGGAGGC TGCATCTTCC
CTAACCTCTG TCTGGCTTG AGCGTTGACAGAGAAAAGGCT TAGTTCTGAA AACCGATATG TTGTTGGAAG
ATGAGCAGCC AGATCACTGCCTAATCTCGC TTTGCTGTCT GTGATGTAGA TGGTGGTTCC TATCCTGAGA
GCAAGTGTGTTTATTCTTTT GC
(polynucleotide, glucose 6-phosphate dehydrogenase (G6PD))
SEQ ID NO: 39
GAGCCCAGCTACATTCCTCAGCTGCCAAGCACTCGAGACCATCCTGGCCCCTCCAGACCCTGCCTGAGCCCAG
GAGCTGAGTCACCTCCTCCACTCACT
(polynucleotide, tyrosine kinase binding protein (TYROBP))
SEQ ID NO: 40
TGCTGGCTGTAAGTGGTCTCCGTCCTGTCCAGGCCCAGGCCCAGAGCGATTGCAGTTGCTCTACGGTGAGCCC
GGGCGTGCTGGCAGGGATCGTGATGG
(polynucleotide, uridine phosphorylase 1 (UPP1))
SEQ ID NO: 41
ACTGCAGACGTCTGTCCGCCTCCAGCCGCTCTCCTCTGACGGGTCCTGCCTCAGTTGGCGGAATGGCGGCCAC
GGGAGCCAATGCAGAGAAAGCT
(polynucleotide, cytochrome P450 family 2 subfamily B member 7,
pseudogene (CYP2B7P1))
SEQ ID NO: 42
AAACCAGACGCCTTCAATCCTGACCACTTTCTGGATGCCAATGGGGCACTGAAAAAGAATGAAGCTTTTATCC
CCTTCTCCTTAGGGAAGCGGATTTGTC
(polynucleotide, uridine phosphorylase 1 (UPP1))
SEQ ID NO: 43
TTAACAAGAAGCTGGTGCAGGAGCTGTTGCTGTGTTCTGCAGAGCTGAGCGAGTTCACCACAGTGGTGGGGA
ACACCATGTGCACCTTGGACTTCTATGA
(polynucleotide, solute carrier family 4 (anion exchanger), member 1
(Diego blood group) (SLC4A1))
SEQ ID NO: 44
AGCAGGAGGAATATGAAGACCCAGACATCCCCGAGTCCCAGATGGAGGAGCCGGCAGCTCACGACACCGAG
GCAACAGCCACAGACTACCACACCACAT
(polynucleotide, cystatin SN (CST1))
SEQ ID NO: 45
AAGGCCACCAAAGATGACTACTACAGACGTCCGCTGCGGGTACTAAGAGCCAGGCAACAGACCGTTGGGGG
GGTGAATTACTTCTTCGACGTAGAGGTG
(polynucleotide, glutaldehyde phosphate dehydrogenase (GAPDH))
SEQ ID NO: 46
TCCTGCACCACCAACTGCTTAGCACCCCTGGCCAAGGTCATCCATGACAACTTTGGTATCGTGGAAGGACTCAT
GACCACAGTCCATGCCA
(polynucleotide, protamine 1 (PRM1))
SEQ ID NO: 47
CTGCTCTGAGCATTCAGGCCAAGCCCATCCTGCACCATGGCCAGGTACAGATGCTGTCGCAGCCAGAGCCGG
AGCAGATATTACCGCCAGAGACAAA
(polynucleotide, uridine phosphorylase 1 (UPP1))
SEQ ID NO: 48
TAGCAGACACAATTTCCCAGCCTTGTTTGGAGATGTGAAGTTTGTGTGTGTTGGTGGAAGCCCCTCCCGGATG
AAAGCCTTCATCAGGTGCGTTG
(polynucleotide, beta-actin (ACTB))
SEQ ID NO: 49
ATCCTCACCCTGAAGTACCCCATCGAGCACGGCATCGTCACCAACTGGGACGACATGGAGAAAATCTGGCACC
ACACCTTCTACAATGAGCTGC
(polynucleotide, beta-actin (ACTB))
SEQ ID NO: 50
GACCTTCAACACCCCAGCCATGTACGTTGCTATCCAGGCTGTGCTATCCCTGTACGCCTCTGGCCGTACCACTG
GCATCGTGATGGACT
(polynucleotide, follicular dendritic cell secreted protein (FDCSP))
SEQ ID NO: 51
TTACCCATATCCATTTCGCCCACTTCCACCAATTCCATTTCCAAGATTTCCATGGTTTAGACGTAATTTTCCTATT
CCAATACCTGAATCTGCCC
(polynucleotide, spermatogenesis associated 42 (SPATA42))
SEQ ID NO: 52
TGGGAATCTGATGGACTCAATTAAGAATTTCTACAGATGGGAAAACCAAAACTCCTTAGTGGCAAGAGGCCA
AAGATGGTCAGCGAATTGTTGTTTCC
(polynucleotide, beta-hemoglobin (HBB))
SEQ ID NO: 53
CTGCACTGTGACAAGCTGCACGTGGATCCTGAGAACTTCAGGCTCCTGGGCAACGTGCTGGTCTGTGTGCTGG
CCCATCACTTTGGCAAAGA
(polynucleotide, homeobox 13 (HOXA13))
SEQ ID NO: 54
CCATTGTAAACATCTGCTTGTCCTTCTTAGGTCGCCATTCCCTTTGCATGTTAAGCGTCTGCTCAGGTAAATCTT
AGTGAAATTCCTACCGTTGTTGTAC
(polynucleotide, protamine 2 (PRM2))
SEQ ID NO: 55
GAACATGCAGAAGGCACTAAGCTTCCTGGGCCCCTCACCCCCAGCTGGAAATTAAGAAAAAGTCGCCCGAAA
CACCAAGTGAGGCCATAGCAATTC
(polynucleotide, kallikrein related peptidase 2 (KLK2))
SEQ ID NO: 56
CACAGGTGTATGCCAATGTTTCTGAAATGGGTATAATTTCGTCCTCTCCTTCGGAACACTGGCTGTCTCTGAAG
ACTTCTCGCTCAGTTTCAGTGA
(polynucleotide, plasminogenin activator urokinase receptor (PLAUR))
SEQ ID NO: 57
AAAGCTATATGGTAAGAGGCTGTGCAACCGCCTCAATGTGCCAACATGCCCACCTGGGTGACGCCTTCAGCAT
GAACCACATTGATGTCTCCTGCTGTA
(polynucleotide, pro-platelet basic protein (chemokine (C-X-C motif)
ligand 7) (PPBP))
SEQ ID NO: 58
TAGACAACTGGACACTCAGGACCACGCCATGGAGGAGCTGCAGGATGATTATGAAGACATGATGGAGGAGA
ATCTGGAGCAGGAGGAATATGAAG
(polynucleotide, protease, serine 21 (PRSS21))
SEQ ID NO: 59
CTATGACATTGCCTTGGTGAAGCTGTCTGCACCTGTCACCTACACTAAACACATCCAGCCCATCTGTCTCCAGG
CCTCCACATTTGAGTTTGAGAA
(polynucleotide, cystatin E/M (CST6))
SEQ ID NO: 60
AACAGCATCTACTACTTCCGAGACACGCACATCATCAAGGCGCAGAGCCAGCTGGTGGCCGGCATCAAGTACT
TCCTGACGATGGAGATGG
(polynucleotide, small proline rich protein 2G (SPRR2G))
SEQ ID NO: 61
GACTCCATCATCTTCCCTTCAGCTGTAGTGGGAGGCTGCATCTTCCCTAACCTCTGTCTGGCTTGAGCGTTGAC
AGAGAAAAGGCTTAGTTCTGA
(polynucleotide, homeobox 11 (HOXA11))
SEQ ID NO: 62
CCATTGAATCTCCTTTGCCTCCCTGTGTTAAGAAATGTCTGTTGGCTCCATTTGTACTGGGAGTGTTGGCCTGTC
CTCAATTCTGGTTCTTAC
(polynucleotide, defensin beta 4A (DEFB4A))
SEQ ID NO: 63
GGACCTTTATAAGGTGGAAGGCTTGATGTCCTCCCCAGACTCAGCTCCTGGTGAAGCTCCCAGCCATCAGCCA
TGAGGGTCTTGTATCTC
(polynucleotide, chemokine (C-C motif) ligand 27 (CCL27))
SEQ ID NO: 64
GTCACAGTGGTTTGAGCACCAAGAGAGAAAGCTCCATGGGACTCTGCCCAAGCTGAATTTTGGGATGCTAAG
GAAAATGGGCTGAAGC
(polynucleotide, uromodulin like 1 (UMODL1))
SEQ ID NO: 65
TTTCTAGACAACTGCTTCACGAGGTCGAGAGCTCCTTCCCACCAGTGGTGTCTGACTTGTACCGAAGTGGGAA
GCTGAGAATGCAGATC
(polynucleotide, semenogelin 1 (SEMG1))
SEQ ID NO: 66
GAAAATGCAAAAGGAGAGTCTGGCCAATCTACAAATAGAGAACAAGACCTACTCAGTCATGAACAAAAAGGC
AGACACCAACATGGATCTCA
(polynucleotide, statherin (STATH))
SEQ ID NO: 67
GAACAACCACTATACCCACAACCATACCAACCACAATACCAACAATATACCTTTTAATATCATCAGTAACTGCA
GGACATGATTATTGAGG
(polynucleotide, ubiquitin conjugating enzyme (UBE2D2))
SEQ ID NO: 68
TTCTTGACAATTCATTTCCCAACAGATTACCCCTTCAAACCACCTAAGGTTGCATTTACAACAAGAATTTATCAT
CCAAATATTAACAGTAATGGCAGC
(polynucleotide, loricrin (LOR))
SEQ ID NO: 69
GCAAATCCTTCATGTCTTAACCTACCTGGAAGAAGCCATTGAGCTCTCCGGCTGCATCTAGTTCTGCTGTTTAG
CCTCTTTGGTTTCTGTACA
(polynucleotide, microseminoprotein beta (MSMB))
SEQ ID NO: 70
GTCTATAAGGAGTCCTGCTTATCACAATGAATGTTCTCCTGGGCAGCGTTGTGATCTTTGCCACCTTCGTGACT
TTATGCAATGCATCATGCTAT
(polynucleotide, keratin 9 (KRT9))
SEQ ID NO: 71
CTGATGGCCCTCAAGAAGAATCATAAGGAGGAGATGAGTCAGCTGACTGGGCAGAACAGTGGAGATGTCAA
TGTGGAGATAAACGTTGC
(polynucleotide, hemoglobin delta (HBD))
SEQ ID NO: 72
CCATGGTGCATCTGACTCCTGAGGAGAAGACTGCTGTCAATGCCCTGTGGGGCAAAGTGAACGTGGATGCAG
TTGGTGG
(polynucleotide, proline-rich protein BstNI subfamily 4 (PRB4))
SEQ ID NO: 73
AGCAGGAGGCAATCCCCAGCAGCCTCAGGCACCTCCTGCTGGAAAGCCCCAGGGGCCACCTCCACCTCCTCAA
GGGG
(polynucleotide, transcription elongation factor SII (TCEA))
SEQ ID NO: 74
TCTGTAATGAATGTGGAAATCGATGGAAGTTCTGTTGAGTTGGAAGAATTGGCAAAATATCTGGACCATTAAG
AAAACGGATTTTGTAACTAGCT
(polynucleotide, myozenin 1 (MYOZ1))
SEQ ID NO: 75
ATCTTGGTTCCAGGGTCTCAGTACCCCCTGTGCCATTTGAGCTGCTTGCGCTCATCATCTCTATTAATAACCAAC
TTCCCTCCC
(polynucleotide, aquaporin 6 (AQP6))
SEQ ID NO: 76
TCCCAATAGGTCTTTATTCCTCAATCCTCCAAATGCTCTGGAGAGGCCCCCACCCTTGAGAAGAACTGACACAG
AGAAGAACATTTTCTCAGG
(polynucleotide, proline-rich protein BstNI subfamily 4 (PRB4))
SEQ ID NO: 77
AAACCAGTCCCAAGGTCCCCCACCTCCTCCAGGAAAGCCAGAAGGACGACCCCCACAAGGAGGCAACCAGTC
CCAA
(polynucleotide, semenogelin 2 (SEMG2))
SEQ ID NO: 78
TGAAGTCCATCATCCTCTTTGTCCTTTCCCTGCTCCTTATCTTGGAGAAGCAAGCAGCTGTGATGGGACAAAAA
GGTGGATCAAAAGG
(polynucleotide, glucose 6-phosphate dehydrogenase (G6PD))
SEQ ID NO: 79
CATCTTCCACCAGCAGTGCAAGCGCAACGAGCTGGTGATCCGCGTGCAGCCCAACGAGGCCGTGTACACCAA
GATGATGA
(polynucleotide, tyrosine kinase binding protein (TYROBP))
SEQ ID NO: 80
TGACAGTCAGCAACATGATACCTGGATCCAGCCATTCCTGAAGCCCACCCTGCACCTCATTCCAACTCCTACCG
CGATACAGA
(polynucleotide, matrix metallopeptidase 11 (MMP11))
SEQ ID NO: 81
CCTTCTACACCTTTCGCTACCCACTGAGTCTCAGCCCAGATGACTGCAGGGGCGTTCAACACCTATATGGCCAG
CCCTGG
(polynucleotide, metallothionein 1 (MT1X))
SEQ ID NO: 82
CTGTGCCAAGTGTGCCCAGGGCTGCATCTGCAAAGGGACGTCAGACAAGTGCAGCTGCTGTGCCTGATGCCAG
(polynucleotide, matrix metallopeptidase 10 (MMP10))
SEQ ID NO: 83
ACCTGGGCTTTATGGAGATATTCACTTTGATGATGATGAAAAATGGACAGAAGATGCATCAGGCACCAATTTA
TTCCTCGTTG
(polynucleotide, ubiquitin conjugating enzyme (UBE2D2))
SEQ ID NO: 84
AAGACAGGCAATCCCTCCGGCTGTCCGACCAAGAGAGGCCGGCCGAGCCCGAGGCTTGGGCTTTTGCTTTCTG
(polynucleotide, transition protein 1 (TNP1))
SEQ ID NO: 85
AAGACAGGCAATCCCTCCGGCTGTCCGACCAAGAGAGGCCGGCCGAGCCCGAGGCTTGGGCTTTTGCTTTCTG
(polynucleotide, transition protein 1 (TNP1))
SEQ ID NO: 86
CAAGGAGACCTGATGTTAGATCAAAGCCAGAGAGGAGCCTATGGAATGTGGATCAAATGCCAGTTGTGACGA
AATGAGG
(polynucleotide, ro-associated Y5 (RNY5))
SEQ ID NO: 87
TCCGAGTGTTGTGGGTTATTGTTAAGTTGATTTAACATTGTCTCCCCCCACAACCGCGCTTGACTAGCTTGCTGT
TT
(polynucleotide, stanniocalcin 1 (STC1))
SEQ ID NO: 88
GCATGAAACTAGGCTTCTGTAATCAATATCCCAACATTCTGCAATGGCAGCATTCCCACCAACAAAATCCATGT
GAC
(polynucleotide, delta -aminolevulinate synthase (ALAS2))
SEQ ID NO: 89
TCAACAGCAAGCTCTGTGATCTCCTGCTCTCCAAGCATGGCATCTATGTGCAGGCCATCAACTACCCAACTGTCC
(polynucleotide, matrix metallopeptidase 3 (MMP3))
SEQ ID NO: 90
GATTAATGGAGATGCCCACTTTGATGATGATGAACAATGGACAAAGGATACAACAGGGACCAATTTATTTCTC
GTTGC
(polynucleotide, glycophorin A (GYPA))
SEQ ID NO: 91
TGATGGCTGGTGTTATTGGAACGATCCTCTTAATTTCTTACGGTATTCGCCGACTGATAAAGAAAAGCCCATCT
GAT
(polynucleotide, small nucleolar RNA, H/ACA box 35 (SNORA35))
SEQ ID NO: 92
GTGCAAAAGCAAATCCCTCTCAAAGCTGGGAGAGTCACACCGTGGGCTACTCCTGCATGCAGCTGGGTACAT
AT
(polynucleotide, transglutaminase 4 (TGM4))
SEQ ID NO: 93
AAGATTGTTCTCATCACCAAGTAGCCTTGTCTGATGCTGTGGAGCCTTAGTTGAGATTTCAGCATTTCCTACCTT
GT
(polynucleotide, histatin 3 (HTN3))
SEQ ID NO: 94
TTCACATCGAGGCTATAGATCAAATTATCTGTATGACAATTGATATCTTCAGTAATCACGGGGCATGATTATG
(polynucleotide, transcription elongation factor SII (TCEA))
SEQ ID NO: 95
AGGATCTCAAATTGAAGAAGCTATATATCAAGAAATAAGGAATACAGACATGAAATACAAAAATAGAGTACG
AAGTAGGATATC
(polynucleotide, Haemoglobin delta (HBD)
SEQ ID NO: 96
ACTGCTGTCAATGCCCTGTG
(polynucleotide, Haemoglobin delta (HBD)
SEQ ID NO: 97
ACCTTCTTGCCATGAGCCTT
(polynucleotide, Solute carrier family 4 (anion exchanger), member 1
(Diego blood group) (SLC4A1))
SEQ ID NO: 98
AACTGGACACTCAGGACCAC
(polynucleotide, Solute carrier family 4 (anion exchanger), member 1
(Diego blood group) (SLC4A1))
SEQ ID NO: 99
GGATGTCTGGGTCTTCATATTCCT
(polynucleotide, Transition protein 1 (during histone to protamine
replacement) (TNP1))
SEQ ID NO: 100
GATGACGCCAATCGCAATTACC
(polynucleotide, Transition protein 1 (during histone to protamine
replacement) (TNP1))
SEQ ID NO: 101
CCTTCTGCTGTTCTTGTTGCTG
(polynucleotide, Kallikrein-related peptidase 2 (KLK2))
SEQ ID NO: 102
CAGTCATGGATGGGCACACT
(polynucleotide, Kallikrein-related peptidase 2 (KLK2))
SEQ ID NO: 103
ACCCTCTGGCCTGTGTCTTC
(polynucleotide, Matrix metallopeptidase 3 (MMP3))
SEQ ID NO: 104
CCATGCCTATGCCCCTG
(polynucleotide, Matrix metallopeptidase 3 (MMP3))
SEQ ID NO: 105
GTCCCTGTTGTATCCTTTGTCC
(polynucleotide, Stanniocalcin 1 (STC1))
SEQ ID NO: 106
TGCCCAATCACTTCTCCAACAG
(polynucleotide, Stanniocalcin 1 (STC1))
SEQ ID NO: 107
TTCTCCATCAGGCTGTCTCTG

1. A method for determing the type of a biological sample, comprising the steps of detecting RNA form the sample associated with any one or more of HBD, SLC4A1, TNP1, KLK2, MMP3 and STC1, M and establising whether the sample is circulatory blood, Spermatozoa, seminal fluid, or menstral fluid.

2. The method of claim 1, comprising detecting RNA for one or more markers asscoiated with sample type.

3. The method of claim 1 comprising detecting an RNA associated with one or more of SEQ ID Nos: 1-95.

4. The method of claim 1 comprising determining if the biological sample is circulatory blood, further comprising the step of detecting RNA associated with HBD, and/or SLC4A1.

5. The method of and one of claim 1 comprising determining if the use of any one the primer pairs of SEQ ID Nos: 96 and 97, and/or 98 and 99.

6. The method of claim 4, further comprising detecting RNA associated with any one of HBB, PPBP and/or GYPA.

7. The method of claim 1 comprising determining if the biological sample is Spermatozoa, further comprising the step of detecting RNA associated with TNP1.

8. The method of claim 1, further comprising the use of the primer pair of SEQ ID Nos: 100 and 101.

9. The method of claim 7, further comprising detecting RNA associated with any one of PRM2, SPATA42, and/or PRM1

10. The method of claim 1 comprising determining if the biological sample is seminal fluid, further comprising the step of detecting RNA associated KLK2.

11. The method of claim 1, further comprising determining the use of the primer pair of SEQ ID Nos: 102 and 103.

12. The method of claim 10, further comprising detecting RNA associated with any one of SEMG1, MSMB, SMEG2 and/or TGM4.

13. The method of claim 1 comprising determining if the biological sample is menstrual fluid, further comprising the step of detecting RNA associated with STC1 and/or MMP3.

14. The method of claim 1, further comprising determining if the use of any one the primer pairs of SEQ ID Nos: 106 and 107, and/or 104 abd 105.

15. The method of claim 10, further comprising detecting RNA associated with any one of PLAUR, MIMP11, and/or MMP10.

16. The method of claim 1, wherein the detection of RNA comprises the use of multiplex PCR, probe analysis and/or micro arrays.

17. A kit for premforming a method according to claim 1, further comprising using at least one primer of Seq Id Nos: 96 to 107.

18. A kit for premforming a method according to claim 1, comprising at least one probe specific for any one or more of of Seq Id Nos: 1 to 95.

19. An isolated sequence of anyone of Seq Id Nos: 96 to 107.


SEQUENCE LISTING (107 sequences)

SEQ ID NO: 1 - DNA, 105 bp, Homo sapiens
cacactgagtgagctgcactgtgacaagctgcacgtggatcctgagaacttcaggctcctgggcaacgtgctggtctgtgtgctggcccatcactttggcaaaga

SEQ ID NO: 2 - DNA, 173 bp, Homo sapiens
gaaccagagataacactcattatttttggggtgatggctggtgttattggaacgatcctcttaatttcttacggtattcgccgactgataaagaaaagcccatctgatgtaaaacctctcccctcacctgacacagacgtgcctttaagttctgttgaaatagaaaatccaga

SEQ ID NO: 3 - DNA, 432 bp, Homo sapiens
gacatcatctctggaactcttggcaaggcctttggctgtgtgggcggctacattgccagcacccgtgacttggtggacatggtgcgctcctatgctgcaggcttcatctttaccacttctctgccccccatggtgctctctggagctctagaatctgtgcggctgctcaagggagaggagggccaagccctgaggcgagcccaccagcgcaatgtcaagcacatgcgccagctactcatggacaggggccttcctgtcatcccctgccccagccacatcatccccatccgggtgggcaatgcagcactcaacagcaagctctgtgatctcctgctctccaagcatggcatctatgtgcaggccatcaactacccaactgtcccccggggtgaagagctcctgcgcttggcaccctccccccaccacagccct

SEQ ID NO: 4 - DNA, 121 bp, Homo sapiens
atgatggaggagaatctggagcaggaggaatatgaagacccagacatccccgagtcccagatggaggagccggcagctcacgacaccgaggcaacagccacagactaccacaccacatcac

SEQ ID NO: 5 - DNA, 243 bp, Homo sapiens
gaggctcgtgagcagggacccgcggtgcgggttatgctgggggctcagatcaccgtagacaactggacactcaggaccacgccatggaggagctgcaggatgattatgaagacatgatggaggagaatctggagcaggaggaatatgaagacccagacatccccgagtcccagatggaggagccggcagctcacgacaccgaggcaacagccacagactaccacaccacatcacacccgggta

SEQ ID NO: 6 - DNA, 103 bp, Homo sapiens
caccatggtgcatctgactcctgaggagaagactgctgtcaatgccctgtggggcaaagtgaacgtggatgcagttggtggtgaggccctgggcagattactg

SEQ ID NO: 7 - DNA, 75 bp, Homo sapiens
gcactgtgacaagctgcacgtggatcctgagaacttcaggctcttgggcaatgtgctggtgtgtgtgctggcccg

SEQ ID NO: 8 - DNA, 124 bp, Homo sapiens
gcccaacgcgctgtccgccctgagcgacctgcacgcgcacaagcttcgggtggacccggtcaacttcaagctcctaagccactgcctgctggtgaccctggccgcccacctccccgccgagttc

SEQ ID NO: 9 - DNA, 630 bp, Homo sapiens
gaaaggacagtaatctcattgttaaaaaaatccaaggaatgcagaagttccttgggttggaggtgacagggaagctagacactgacactctggaggtgatgcgcaagcccaggtgtggagttcctgacgttggtcacttcagctcctttcctggcatgccgaagtggaggaaaacccaccttacatacaggattgtgaattatacaccagatttgccaagagatgctgttgattctgccattgagaaagctctgaaagtctgggaagaggtgactccactcacattctccaggctgtatgaaggagaggctgatataatgatctcttttgcagttaaagaacatggagacttttactcttttgatggcccaggacacagtttggctcatgcctacccacctggacctgggctttatggagatattcactttgatgatgatgaaaaatggacagaagatgcatcaggcaccaatttattcctcgttgctgctcatgaacttggccactccctggggctctttcactcagccaacactgaagctttgatgtacccactctacaactcattcacagagctcgcccagttccgcctttcgcaagatgatgtgaatggcattcagtctctctacg

SEQ ID NO: 10 - DNA, 196 bp, Homo sapiens
acagacctgctgcaggtggcagcccatgaatttggccacgtgctggggctgcagcacacaacagcagccaaggccctgatgtccgccttctacacctttcgctacccactgagtctcagcccagatgactgcaggggcgttcaacacctatatggccagccctggcccactgtcacctccaggaccccagccctgg

SEQ ID NO: 11 - DNA, 310 bp, Homo sapiens
gattgtgaattatacaccagatttgccaaaagatgctgttgattctgctgttgagaaagctctgaaagtctgggaagaggtgactccactcacattctccaggctgtatgaaggagaggctgatataatgatctcttttgcagttagagaacatggagacttttacccttttgatggacctggaaatgttttggcccatgcctatgcccctgggccagggattaatggagatgcccactttgatgatgatgaacaatggacaaaggatacaacagggaccaatttatttctcgttgctgctcatgaaatt

SEQ ID NO: 12 - DNA, 403 bp, Homo sapiens
tcctggagcttgaaaatctgccgcagaatggccgccagtgttacagctgcaaggggaacagcacccatggatgctcctctgaagagactttcctcattgactgccgaggccccatgaatcaatgtctggtagccaccggcactcacgaaccgaaaaaccaaagctatatggtaagaggctgtgcaaccgcctcaatgtgccaacatgcccacctgggtgacgccttcagcatgaaccacattgatgtctcctgctgtactaaaagtggctgtaaccacccagacctggatgtccagtaccgcagtggggctgctcctcagcctggccctgcccatctcagcctcaccatcaccctgctaatgactgccagactgtggggaggcactctcctctggacctaaac

SEQ ID NO: 13 - DNA, 496 bp, Homo sapiens
agacacagtcagcacaatcagagacagcctgatggagaaaattgggcctaacatggccagcctcttccacatcctgcagacagaccactgtgcccaaacacacccacgagctgacttcaacaggagacgcaccaatgagccgcagaagctgaaagtcctcctcaggaacctccgaggtgaggaggactctccctcccacatcaaacgcacatcccatgagagtgcataaccagggagaggttattcacaacctcaccaaactagtatcattttaggggtgttgacacaccagttttgagtgtactgtgcctggtttgatttttttaaagtagttcctattttctatcccccttaaagaaaattgcatgaaactaggcttctgtaatcaatatcccaacattctgcaatggcagcattcccaccaacaaaatccatgtgaccattctgcctctcctcaggagaaagtaccctcttttaccaacttcctctgccatgt

SEQ ID NO: 14 - DNA, 369 bp, Homo sapiens
ttgcctaacacaggcagaattggccagctacttgtctgcaattgtatcttcaagaataccctggccatccctttgactgacgtcaagttctctttggaaagcctgggcatctcctcactacagacctctgaccatgggacggtgcagcctggtgagaccatccaatcccaaataaaatgcaccccaataaaaactggacccaagaaatttatcgtcaagttaagttccaaacaagtgaaagagattaatgctcagaagattgttctcatcaccaagtagccttgtctgatgctgtggagccttagttgagatttcagcatttcctaccttgtgcttagctttcagattatggatgattaaatttgatga

SEQ ID NO: 15 - DNA, 134 bp, Homo sapiens
atgaagtccatcatcctctttgtcctttccctgctccttatcttggagaagcaagcagctgtgatgggacaaaaaggtggatcaaaaggccaattgccaagcggatcttcccaatttccacatggacaaaaggg

SEQ ID NO: 16 - DNA, 221 bp, Homo sapiens
aaatccaggcaccaaatcctaagcaagagccatggcatggtgaaaatgcaaaaggagagtctggccaatctacaaatagagaacaagacctactcagtcatgaacaaaaaggcagacaccaacatggatctcatgggggattggatattgtaattatagagcaggaagatgacagtgatcgtcatttggcacaacatcttaacaacgaccgaaacccatta

SEQ ID NO: 17 - DNA, 139 bp, Homo sapiens
gtacctgtctataaggagtcctgcttatcacaatgaatgttctcctgggcagcgttgtgatctttgccaccttcgtgactttatgcaatgcatcatgctatttcatacctaatgagggagttccaggagattcaaccag

SEQ ID NO: 18 - DNA, 101 bp, Homo sapiens
actgggaatctgatggactcaattaagaatttctacagatgggaaaaccaaaactccttagtggcaagaggccaaagatggtcagcgaattgttgtttccg

SEQ ID NO: 19 - DNA, 179 bp, Homo sapiens
tgctcacaggttggctggctcagccaaggtggtgccctgctctgagcattcaggccaagcccatcctgcaccatggccaggtacagatgctgtcgcagccagagccggagcagatattaccgccagagacaaagaagtcgcagacgaaggaggcggagctgccagacacggaggagagc

SEQ ID NO: 20 - DNA, 80 bp, Homo sapiens
ttcacatcgaggctatagatcaaattatctgtatgacaattgatatcttcagtaatcacggggcatgattatggaggttt

SEQ ID NO: 21 - DNA, 187 bp, Homo sapiens
gtatggcccttatcagccagttccagaacaaccactatacccacaaccataccaaccacaataccaacaatataccttttaatatcatcagtaactgcaggacatgattattgaggcttgattggcaaatacgacttctacatccatattctcatctttcataccatatcacactactaccactttt

SEQ ID NO: 22 - DNA, 191 bp, Homo sapiens
atcagtgacagcgatgaattagcttcagggttttttgtgttcccttacccatatccatttcgcccacttccaccaattccatttccaagatttccatggtttagacgtaattttcctattccaatacctgaatctgcccctacaactccccttcctagcgaaaagtaaacaagaaggaaaagtcacgataa

SEQ ID NO: 23 - DNA, 132 bp, Homo sapiens
aaaccagtcccaaggtcccccacctcctccaggaaagccagaaggacgacccccacaaggaggcaaccagtcccaaggtcccccacctcatccaggaaagccagaaagaccacccccacaaggaggaaacca

SEQ ID NO: 24 - DNA, 163 bp, Homo sapiens
tggaaagccacaaggcccacccccagcaggaggcaatccccagcagcctcaggcacctcctgctggaaagccccaggggccacctccacctcctcaagggggcaggccacccagacctgcccagggacaacagcctccccagtaatctaggattcaatgacag

SEQ ID NO: 25 - DNA, 61 bp, Homo sapiens
ttggctcctgtgcctgtgccggctcctgcaaatgcaaagagtgcaaatgcacctcctgcaa

SEQ ID NO: 26 - DNA, 133 bp, Homo sapiens
gctcctgctgccctgtgggctgtgccaagtgtgcccagggctgcatctgcaaagggacgtcagacaagtgcagctgctgtgcctgatgccaggacagctgtgctctcagatgtaaatagagcaacctatataa

SEQ ID NO: 27 - DNA, 177 bp, Homo sapiens
gcttggtgaggtgactcgcggtcgcgggtgactcgccggcaggacactgcctggaacgcctggagcgcctcccactgcagacgtctgtccgcctccagccgctctcctctgacgggtcctgcctcagttggcggaatggcggccacgggagccaatgcagagaaagctgaaagtcac

SEQ ID NO: 28 - DNA, 118 bp, Homo sapiens
tttcaatctcaccactagcagacacaatttcccagccttgtttggagatgtgaagtttgtgtgtgttggtggaagcccctcccggatgaaagccttcatcaggtgcgttggtgcagag

SEQ ID NO: 29 - DNA, 353 bp, Homo sapiens
gcagattgtcctggggaagcgggtcatccggaaaacggaccttaacaagaagctggtgcaggagctgttgctgtgttctgcagagctgagcgagttcaccacagtggtggggaacaccatgtgcaccttggacttctatgaagggcaaggccgtctggatggggctctctgctcctacacggagaaggacaagcaggcgtatctggaggcagcctatgcagccggcgtccgcaatatcgagatggagtcctcggtgtttgccgccatgtgcagcgcctgcggcctccaagcggccgtggtgtgtgtcaccctcctgaaccgcctggaaggggaccagatcagcagccctcgca

SEQ ID NO: 30 - DNA, 69 bp, Homo sapiens
cctgatttctgcagctctgtgtgaaggtgcagttttgccaaggagtgctaaagaacttagatgtcagtg

SEQ ID NO: 31 - DNA, 49 bp, Homo sapiens
gcgccaacacagaaattattgtaaagctttctgatggaagagagctctg

SEQ ID NO: 32 - DNA, 192 bp, Homo sapiens
agtgggagaatcccaaaggccttttccctccttcctgagcctccgggcaaggagggagggatcttggttccagggtctcagtaccccctgtgccatttgagctgcttgcgctcatcatctctattaataaccaacttccctcccccactgccagtgctgcccccacgcctgcccagctcgtgttctccggtc

SEQ ID NO: 33 - DNA, 136 bp, Homo sapiens
caggacctttataaggtggaaggcttgatgtcctccccagactcagctcctggtgaagctcccagccatcagccatgagggtcttgtatctcctcttctcgttcctcttcatattcctgatgcctcttccaggtgt

SEQ ID NO: 34 - DNA, 166 bp, Homo sapiens
ggggggacttgaaccctgcagcaggctcctgctcctgcctctcctgctggctgtaagtggtctccgtcctgtccaggcccaggcccagagcgattgcagttgctctacggtgagcccgggcgtgctggcagggatcgtgatgggagacctggtgctgacagtgctc

SEQ ID NO: 35 - DNA, 123 bp, Homo sapiens
gcccgaatcatgacagtcagcaacatgatacctggatccagccattcctgaagcccaccctgcacctcattccaactcctaccgcgatacagacccacagagtgccatccctgagagaccaga

SEQ ID NO: 36 - DNA, 328 bp, Homo sapiens
cactgctctccgtgacccacactactttgaaaaaccagacgccttcaatcctgaccactttctggatgccaatggggcactgaaaaagaatgaagcttttatccccttctccttagggaagcggatttgtcttggtgaaggcattgcccgtgcggaattgttcctcttcttcaccaccatcctccagaacttctccgtggccagccccgtggctcctgaagacatcgatctgacaccccaggagtgtggtgtgggcaaaatacccccaacataccagatctgcttcctgccccgctgaaggggctgagggaagggggtcaaaggattc

SEQ ID NO: 37 - DNA, 72 bp, Homo sapiens
gctcactttggcagcacatataactaaaattggaatgctgcagagaagattagcatggcccctacatttaaa

SEQ ID NO: 38 - DNA, 227 bp, Homo sapiens
tggctcttcttactcccaggactccatcatcttcccttcagctgtagtgggaggctgcatcttccctaacctctgtctggcttgagcgttgacagagaaaaggcttagttctgaaaaccgatatgttgttggaagatgagcagccagatcactgcctaatctcgctttgctgtctgtgatgtagatggtggttcctatcctgagagcaagtgtgtttattcttttgc

SEQ ID NO: 39 - DNA, 99 bp, Homo sapiens
gagcccagctacattcctcagctgccaagcactcgagaccatcctggcccctccagaccctgcctgagcccaggagctgagtcacctcctccactcact

SEQ ID NO: 40 - DNA, 99 bp, Homo sapiens
tgctggctgtaagtggtctccgtcctgtccaggcccaggcccagagcgattgcagttgctctacggtgagcccgggcgtgctggcagggatcgtgatgg

SEQ ID NO: 41 - DNA, 95 bp, Homo sapiens
actgcagacgtctgtccgcctccagccgctctcctctgacgggtcctgcctcagttggcggaatggcggccacgggagccaatgcagagaaagct

SEQ ID NO: 42 - DNA, 100 bp, Homo sapiens
aaaccagacgccttcaatcctgaccactttctggatgccaatggggcactgaaaaagaatgaagcttttatccccttctccttagggaagcggatttgtc

SEQ ID NO: 43 - DNA, 100 bp, Homo sapiens
ttaacaagaagctggtgcaggagctgttgctgtgttctgcagagctgagcgagttcaccacagtggtggggaacaccatgtgcaccttggacttctatga

SEQ ID NO: 44 - DNA, 99 bp, Homo sapiens
agcaggaggaatatgaagacccagacatccccgagtcccagatggaggagccggcagctcacgacaccgaggcaacagccacagactaccacaccacat

SEQ ID NO: 45 - DNA, 99 bp, Homo sapiens
aaggccaccaaagatgactactacagacgtccgctgcgggtactaagagccaggcaacagaccgttgggggggtgaattacttcttcgacgtagaggtg

SEQ ID NO: 46 - DNA, 91 bp, Homo sapiens
tcctgcaccaccaactgcttagcacccctggccaaggtcatccatgacaactttggtatcgtggaaggactcatgaccacagtccatgcca

SEQ ID NO: 47 - DNA, 97 bp, Homo sapiens
ctgctctgagcattcaggccaagcccatcctgcaccatggccaggtacagatgctgtcgcagccagagccggagcagatattaccgccagagacaaa

SEQ ID NO: 48 - DNA, 95 bp, Homo sapiens
tagcagacacaatttcccagccttgtttggagatgtgaagtttgtgtgtgttggtggaagcccctcccggatgaaagccttcatcaggtgcgttg

SEQ ID NO: 49 - DNA, 94 bp, Homo sapiens
atcctcaccctgaagtaccccatcgagcacggcatcgtcaccaactgggacgacatggagaaaatctggcaccacaccttctacaatgagctgc

SEQ ID NO: 50 - DNA, 89 bp, Homo sapiens
gaccttcaacaccccagccatgtacgttgctatccaggctgtgctatccctgtacgcctctggccgtaccactggcatcgtgatggact

SEQ ID NO: 51 - DNA, 95 bp, Homo sapiens
ttacccatatccatttcgcccacttccaccaattccatttccaagatttccatggtttagacgtaattttcctattccaatacctgaatctgccc

SEQ ID NO: 52 - DNA, 98 bp, Homo sapiens
tgggaatctgatggactcaattaagaatttctacagatgggaaaaccaaaactccttagtggcaagaggccaaagatggtcagcgaattgttgtttcc

SEQ ID NO: 53 - DNA, 92 bp, Homo sapiens
ctgcactgtgacaagctgcacgtggatcctgagaacttcaggctcctgggcaacgtgctggtctgtgtgctggcccatcactttggcaaaga

SEQ ID NO: 54 - DNA, 100 bp, Homo sapiens
ccattgtaaacatctgcttgtccttcttaggtcgccattccctttgcatgttaagcgtctgctcaggtaaatcttagtgaaattcctaccgttgttgtac

SEQ ID NO: 55 - DNA, 96 bp, Homo sapiens
gaacatgcagaaggcactaagcttcctgggcccctcacccccagctggaaattaagaaaaagtcgcccgaaacaccaagtgaggccatagcaattc

SEQ ID NO: 56 - DNA, 96 bp, Homo sapiens
cacaggtgtatgccaatgtttctgaaatgggtataatttcgtcctctccttcggaacactggctgtctctgaagacttctcgctcagtttcagtga

SEQ ID NO: 57 - DNA, 99 bp, Homo sapiens
aaagctatatggtaagaggctgtgcaaccgcctcaatgtgccaacatgcccacctgggtgacgccttcagcatgaaccacattgatgtctcctgctgta

SEQ ID NO: 58 - DNA, 95 bp, Homo sapiens
tagacaactggacactcaggaccacgccatggaggagctgcaggatgattatgaagacatgatggaggagaatctggagcaggaggaatatgaag

SEQ ID NO: 59 - DNA, 96 bp, Homo sapiens
ctatgacattgccttggtgaagctgtctgcacctgtcacctacactaaacacatccagcccatctgtctccaggcctccacatttgagtttgagaa

SEQ ID NO: 60 - DNA, 91 bp, Homo sapiens
aacagcatctactacttccgagacacgcacatcatcaaggcgcagagccagctggtggccggcatcaagtacttcctgacgatggagatgg

SEQ ID NO: 61 - DNA, 95 bp, Homo sapiens
gactccatcatcttcccttcagctgtagtgggaggctgcatcttccctaacctctgtctggcttgagcgttgacagagaaaaggcttagttctga

SEQ ID NO: 62 - DNA, 93 bp, Homo sapiens
ccattgaatctcctttgcctccctgtgttaagaaatgtctgttggctccatttgtactgggagtgttggcctgtcctcaattctggttcttac

SEQ ID NO: 63 - DNA, 90 bp, Homo sapiens
ggacctttataaggtggaaggcttgatgtcctccccagactcagctcctggtgaagctcccagccatcagccatgagggtcttgtatctc

SEQ ID NO: 64 - DNA, 88 bp, Homo sapiens
gtcacagtggtttgagcaccaagagagaaagctccatgggactctgcccaagctgaattttgggatgctaaggaaaatgggctgaagc

SEQ ID NO: 65 - DNA, 89 bp, Homo sapiens
tttctagacaactgcttcacgaggtcgagagctccttcccaccagtggtgtctgacttgtaccgaagtgggaagctgagaatgcagatc

SEQ ID NO: 66 - DNA, 92 bp, Homo sapiens
gaaaatgcaaaaggagagtctggccaatctacaaatagagaacaagacctactcagtcatgaacaaaaaggcagacaccaacatggatctca

SEQ ID NO: 67 - DNA, 91 bp, Homo sapiens
gaacaaccactatacccacaaccataccaaccacaataccaacaatataccttttaatatcatcagtaactgcaggacatgattattgagg

SEQ ID NO: 68 - DNA, 99 bp, Homo sapiens
ttcttgacaattcatttcccaacagattaccccttcaaaccacctaaggttgcatttacaacaagaatttatcatccaaatattaacagtaatggcagc

SEQ ID NO: 69 - DNA, 93 bp, Homo sapiens
gcaaatccttcatgtcttaacctacctggaagaagccattgagctctccggctgcatctagttctgctgtttagcctctttggtttctgtaca

SEQ ID NO: 70 - DNA, 95 bp, Homo sapiens
gtctataaggagtcctgcttatcacaatgaatgttctcctgggcagcgttgtgatctttgccaccttcgtgactttatgcaatgcatcatgctat

SEQ ID NO: 71 - DNA, 89 bp, Homo sapiens
ctgatggccctcaagaagaatcataaggaggagatgagtcagctgactgggcagaacagtggagatgtcaatgtggagataaacgttgc

SEQ ID NO: 72 - DNA, 79 bp, Homo sapiens
ccatggtgcatctgactcctgaggagaagactgctgtcaatgccctgtggggcaaagtgaacgtggatgcagttggtgg

SEQ ID NO: 73 - DNA, 77 bp, Homo sapiens
agcaggaggcaatccccagcagcctcaggcacctcctgctggaaagccccaggggccacctccacctcctcaagggg

SEQ ID NO: 74 - DNA, 95 bp, Homo sapiens
tctgtaatgaatgtggaaatcgatggaagttctgttgagttggaagaattggcaaaatatctggaccattaagaaaacggattttgtaactagct

SEQ ID NO: 75 - DNA, 84 bp, Homo sapiens
atcttggttccagggtctcagtaccccctgtgccatttgagctgcttgcgctcatcatctctattaataaccaacttccctccc

SEQ ID NO: 76 - DNA, 93 bp, Homo sapiens
tcccaataggtctttattcctcaatcctccaaatgctctggagaggcccccacccttgagaagaactgacacagagaagaacattttctcagg

SEQ ID NO: 77 - DNA, 76 bp, Homo sapiens
aaaccagtcccaaggtcccccacctcctccaggaaagccagaaggacgacccccacaaggaggcaaccagtcccaa

SEQ ID NO: 78 - DNA, 88 bp, Homo sapiens
tgaagtccatcatcctctttgtcctttccctgctccttatcttggagaagcaagcagctgtgatgggacaaaaaggtggatcaaaagg

SEQ ID NO: 79 - DNA, 80 bp, Homo sapiens
catcttccaccagcagtgcaagcgcaacgagctggtgatccgcgtgcagcccaacgaggccgtgtacaccaagatgatga

SEQ ID NO: 80 - DNA, 83 bp, Homo sapiens
tgacagtcagcaacatgatacctggatccagccattcctgaagcccaccctgcacctcattccaactcctaccgcgatacaga

SEQ ID NO: 81 - DNA, 80 bp, Homo sapiens
ccttctacacctttcgctacccactgagtctcagcccagatgactgcaggggcgttcaacacctatatggccagccctgg

SEQ ID NO: 82 - DNA, 73 bp, Homo sapiens
ctgtgccaagtgtgcccagggctgcatctgcaaagggacgtcagacaagtgcagctgctgtgcctgatgccag

SEQ ID NO: 83 - DNA, 83 bp, Homo sapiens
acctgggctttatggagatattcactttgatgatgatgaaaaatggacagaagatgcatcaggcaccaatttattcctcgttg

SEQ ID NO: 84 - DNA, 73 bp, Homo sapiens
aagacaggcaatccctccggctgtccgaccaagagaggccggccgagcccgaggcttgggcttttgctttctg

SEQ ID NO: 85 - DNA, 73 bp, Homo sapiens; identical to SEQ ID NO: 84

SEQ ID NO: 86 - DNA, 79 bp, Homo sapiens
caaggagacctgatgttagatcaaagccagagaggagcctatggaatgtggatcaaatgccagttgtgacgaaatgagg

SEQ ID NO: 87 - DNA, 77 bp, Homo sapiens
tccgagtgttgtgggttattgttaagttgatttaacattgtctccccccacaaccgcgcttgactagcttgctgttt

SEQ ID NO: 88 - DNA, 77 bp, Homo sapiens
gcatgaaactaggcttctgtaatcaatatcccaacattctgcaatggcagcattcccaccaacaaaatccatgtgac

SEQ ID NO: 89 - DNA, 75 bp, Homo sapiens
tcaacagcaagctctgtgatctcctgctctccaagcatggcatctatgtgcaggccatcaactacccaactgtcc

SEQ ID NO: 90 - DNA, 78 bp, Homo sapiens
gattaatggagatgcccactttgatgatgatgaacaatggacaaaggatacaacagggaccaatttatttctcgttgc

SEQ ID NO: 91 - DNA, 77 bp, Homo sapiens
tgatggctggtgttattggaacgatcctcttaatttcttacggtattcgccgactgataaagaaaagcccatctgat

SEQ ID NO: 92 - DNA, 74 bp, Homo sapiens
gtgcaaaagcaaatccctctcaaagctgggagagtcacaccgtgggctactcctgcatgcagctgggtacatat

SEQ ID NO: 93 - DNA, 77 bp, Homo sapiens
aagattgttctcatcaccaagtagccttgtctgatgctgtggagccttagttgagatttcagcatttcctaccttgt

SEQ ID NO: 94 - DNA, 73 bp, Homo sapiens
ttcacatcgaggctatagatcaaattatctgtatgacaattgatatcttcagtaatcacggggcatgattatg

SEQ ID NO: 95 - DNA, 84 bp, Homo sapiens
aggatctcaaattgaagaagctatatatcaagaaataaggaatacagacatgaaatacaaaaatagagtacgaagtaggatatc

SEQ ID NO: 96 - DNA, 20 bp, Homo sapiens
actgctgtcaatgccctgtg

SEQ ID NO: 97 - DNA, 20 bp, Homo sapiens
accttcttgccatgagcctt

SEQ ID NO: 98 - DNA, 20 bp, Homo sapiens
aactggacactcaggaccac

SEQ ID NO: 99 - DNA, 24 bp, Homo sapiens
ggatgtctgggtcttcatattcct

SEQ ID NO: 100 - DNA, 22 bp, Homo sapiens
gatgacgccaatcgcaattacc

SEQ ID NO: 101 - DNA, 22 bp, Homo sapiens
ccttctgctgttcttgttgctg

SEQ ID NO: 102 - DNA, 20 bp, Homo sapiens
cagtcatggatgggcacact

SEQ ID NO: 103 - DNA, 20 bp, Homo sapiens
accctctggcctgtgtcttc

SEQ ID NO: 104 - DNA, 17 bp, Homo sapiens
ccatgcctatgcccctg

SEQ ID NO: 105 - DNA, 22 bp, Homo sapiens
gtccctgttgtatcctttgtcc

SEQ ID NO: 106 - DNA, 22 bp, Homo sapiens
tgcccaatcacttctccaacag

SEQ ID NO: 107 - DNA, 21 bp, Homo sapiens
ttctccatcaggctgtctctg